(19) United States
(12) Patent Application Publication
(10) Pub. No.: US 2021/0348161 A1
(43) Pub. Date: Nov. 11, 2021

(54) GENE EDITING BASED CANCER TREATMENT

(71) Applicants: FUNDACIÓN DEL SECTOR PÚBLICO ESTATAL CENTRO NACIONAL DE INVESTIGACIONES ONCOLÓGICAS CARLOS III (F.S., Madrid (ES); FUNDACIÓN INSTITUTO DE INVESTIGACION CONTRA LA LEUCEMIA JOSEP CARRERAS, Barcelona (ES)

(72) Inventors: Sandra RODRIGUEZ PERALES, Madrid (ES); Raúl TORRES RUIZ, Madrid (ES); Marta MARTINEZ-LAGE, Madrid (ES)

(21) Appl. No.: 17/286,300

(22) PCT Filed: Oct. 18, 2019
(86) PCT No.: PCT/EP2019/078408

§ 371 Date: Apr. 16, 2021

(30) Foreign Application Priority Data
Oct. 18, 2018 (EP) 18382746.8

(51) Int. Cl. C12N 15/11; C12N 9/22; C12N 15/113; A61P 35/00

(52) U.S. Cl. CPC C12N 15/111; C12N 9/22; C12N 2320/30; A61P 35/00; C12N 2310/20; C12N 15/1135

(57) ABSTRACT

The present invention relates to a method for eliminating cancer cells, wherein said cells comprise a genomic rearrangement which leads either to the expression of a fusion gene not present in non-cancer cells, or to genomic amplifications or rearrangements which lead to the induction of the expression or to the overexpression of a cancer inducing gene, said method comprising: (a) cleaving the genome in at least two sites, said cleavage leading to either a deletion, an inversion, a frameshift, the cleavage without repair and/or an insertion in the genome of said cancer cells, and/or (b) cleaving the expression product of said fusion gene or cancer inducing gene in at least one site.

### FIELD OF THE INVENTION

The present invention belongs to the field of Biomedicine and relates to a gene-editing based cancer treatment where cancer cells are selectively eliminated.

### BACKGROUND OF THE INVENTION

Specific, recurrent chromosomal rearrangements are very common and well-known hallmarks of cancer. Genes affected by chromosome aberrations, in particular translocations, deletions and inversions, fall into two categories: proto-oncogenes that undergo enforced expression as a result of their new chromosomal context, or fusion genes where the breakpoints are within introns of the affected genes on the two involved chromosomes. The latter is the more common consequence of chromosomal translocations, and results in the creation of new chimeric genes consequence of the fusion of the coding sequences of two different genes¹. The introduction of next-generation sequencing (NGS) technologies has dramatically changed the gene fusion landscape providing a radically new means to identify fusions. Using NGS, a plethora of gene fusions (more than 9,000) has now been identified. To date, more than 350 recurrent fusion genes involving more than 300 different genes have been identified¹⁰. Although the products of oncogenic fusion genes are diverse, they can primarily be classified into two groups, transcription factors and tyrosine kinases (TKs).

Fusion genes have critical functions in tumorigenesis and are exceptionally powerful cancer mutations, as they often have multiple effects on a target gene: in a single ‘mutation’ they can dramatically change expression, remove regulatory domains, force oligomerization, change the subcellular location of a protein or join it to novel binding domains. This is reflected clinically in the fact that some neoplasms are classified or managed according to the presence of a particular fusion gene³. Fusion genes are tumour-specific and therefore important targets for therapy: promyelocytic leukaemias that have PML-RARα fusion of the retinoic acid receptor-α are treated with retinoic acid⁴, and the BCR-ABL fusion gene of chronic myeloid leukaemia is the target of the iconic targeted drug Glivec (STI-571)⁵.

There is strong evidence that gene fusions represent important and early steps in the initiation of carcinogenesis. First, they are usually closely correlated with specific tumour phenotypes⁶⁻¹⁰. Second, it has been shown that successful treatment is paralleled by a decrease or eradication of the disease-associated chimera¹¹⁻¹⁴. And finally, silencing fusion transcripts in vitro leads to the reversal of tumorigenicity, decreased proliferation and/or differentiation^{15,16}.

Gene Fusion Products as Tumour Specific Therapeutic Targets

Gene fusions produce tumour-specific molecules because the chimeric RNA and protein product only occurs in the cell with the chromosomal rearrangement (translocation, deletion or inversion). These unique molecules are potential tumour specific therapeutic targets. One important problem of gene fusion products as therapeutic targets is their intracellular location. Although intracellular delivery of therapeutic molecules is challenging, their tumour specificity is an important motivating factor for developing new-targeted therapies. The flow of genetic information from DNA to mRNA and to proteins has several points at which therapeutic reagents could intervene. Several approaches have been developed to target gene fusions, including:

1. —Targeting protein: small molecules, intrabodies and aptamers.

2. —Targeting mRNA: antisense, ribozymes and RNAi.

3. —Targeting DNA: genome editing: The CRISPR-Cas9 systems, which can generate targeted breaks in the genome at any desired location allowing direct gene editing, can be used to target chromosomal DNA breakpoints that create the fusion genes providing a genotype-specific approach to treating human cancers¹⁷. The Cas9 is a DNA endonuclease that can be targeted to a specific 20-bp DNA sequence by a single guide RNA (sgRNA)^{18,19}. Luo's group²⁰, by using Cas9 nickase mediated genome editing, were able to insert HSV1-tk into patient specific chromosomal breakpoints of the fusion genes TMEM-CCDC67 and MAN2A1-FER, found in prostate cancer and hepatocellular carcinoma, respectively. Treatment of tumours bearing these chromosome breakpoints with ganciclovir after induction of HSV1-tk led to cell death in cell culture and to a decrease in tumour size and mortality in mice xenografted with human prostates and liver cancers. Although genotype-specific, this therapy approach relies on a knock-in strategy that nowadays is associated with low efficiencies (0.1-10%). On the other hand, this approach depends on the previous knowledge of the breakpoint sequence which is patient specific what makes necessary a sequencing study of the introns more probably involved in the translocation together with the design and development of new targeting tools (sgRNAs and donor template) for the treatment of each particular patient. This approach could also be associated with wild type cell death events associated with TK random integration.

Working in the same direction of targeting cancer fusion genes that do not exist in normal cells but using a more efficient and no patient specific strategy, the inventors have developed a radically simple, versatile, highly efficient and clinically relevant gene editing approach based on the targeted deletion of a large genomic region containing the fusion oncogene leaving unaltered the exonic regions of their corresponding wild-type alleles. The CRISPR-Cas9 approach is based on an efficient (30-80%) knock-out strategy to selectively destroy cancer cells that harbour recurrent fusion genes whilst sparing the normal counterparts.

WO2016094888 A1 relates to the use of CRISPR and compositions comprising a guide RNA and a Cas protein, specifically for introducing a suicidal gene into in the breakpoint loci of a cancer-specific target sequence which is a fusion gene.

Gene amplification is frequently observed in cancer, especially in solid tumors, and has been thought to contribute to tumor evolution. Gene amplification refers to the somatically acquired increase in copy number of a restricted region of the genome. The amplification is a genomic mechanism that results in overexpression of a dominantly acting cancer gene³¹. These amplified regions, known as amplicons, can span kilobases to tens of megabases and can include multiple oncogenic genes as well as passenger genes in the amplified regions³². Amplification events have classically been linked to the cytogenetic features of double minutes, self-replicating extra-chromosomal elements, or homogenously staining regions where multiple copies of a genomic region or regions are integrated into a chromosome³³. The number of copies of a DNA sequence that constitutes a genomic amplification is variously described but generally considered greater than 4 or 5-fold relative to an adjacent non-amplified marker on the same chromosome. In a diploid genome this would be equivalent to more than 8 copies. TCGA analysis have identified 461 genes statistically amplified in 14 cancer types³⁴. However, some of the genes identified as cancer amplified genes may be passenger genes in the amplicons. Copy number versus expression analysis revealed 73 potential driver genes³¹. Several targeted therapies have been developed to inhibit the functions of amplified oncogenes. These therapies include molecular targeted therapies such as tyrosine kinase inhibitors (TKIs), which include gefitinib and erlotinib for EGFR; or monoclonal antibodies such as trastuzumab for ERBB2 or cetuximab for EGFR³⁵.

However, there is a need for a more robust and specific therapy both against fusion protein related cancers and amplification related cancers. There is still a need of therapeutic tools against cancer which are universal and not patient specific, and which are more efficient and act only on cancer cells, minimizing the side effects of the treatment.

### DESCRIPTION OF THE INVENTION

The present invention provides a way to eliminate cancer cells specifically using endonuclaease(s) that cleave the genome at specific sites, which results in a selective elimination of the cancer inducing gene and thereby elimination of the cancer cells.

The cleavage is directed to the genomic rearrangement which leads either to the expression of a fusion gene absent in non-cancer cells, or to genomic amplifications or rearrangements which lead to the induction of the expression or to the overexpression of a cancer inducing gene. The inventors have found a simple and straightforward way to design a treatment which is universal (not patient specific), since it does not depend on the specific sequence (breakpoint) where the genomic rearrangement is occurring. For those cancers where there is a fusion gene and fusion protein, the present invention allows the truncation or the elimination of the fusion protein, which in turn leads to the death of the cancer cell. But more importantly, the present invention provides a therapy with minimal side effects since the modification of the coding regions of the genome will only take place in cells carrying the genomic rearrangement, i.e. in cancer cells. For those cancers where the cancer cells comprise an amplified region including one or more oncogenes, the method of the present invention is extremely robust, since the cancer cell genome is damaged in an irreversible way, and the cancer cell is doomed to cell death, while normal cells remain unaffected.

Thus, in a first aspect, the present invention relates to a method for eliminating cancer cells, wherein said cells comprise a genomic rearrangement which leads either to the expression a fusion gene not present in non-cancer cells, or to genomic amplifications or rearrangements which lead to the induction of the expression or to the overexpression of a cancer inducing gene, said method comprising: (a) cleaving the genome in at least two sites, said cleavage leading to either a deletion, an inversion, a frameshift, the cleavage without repair and/or an insertion in the genome of said cancer cells, or (b) cleaving the expression product of said fusion gene or cancer inducing gene in at least one site.

As used herein, the term “cleaving”, “cleave” or “cleavage” means that both DNA chains or strands are cut when it is referred to the genome, which is double stranded DNA. When said term is referred to a DNA or RNA molecule which is double stranded, it means that both chains or strands are cut. When said term is referred to a DNA or RNA molecule which is single stranded, it means that only one chain or strand is cut. Upon genome cleavage, when a double stranded molecule is cut, both sticky and blunt ends may be generated as a result of the cleavage. When the method of the invention is used to eliminate cancer cells where the genomic rearrangement leads to a genomic amplification, the cleavage leads to the genome damage where the cleaved DNA cannot be repaired at the cleavage sites. Therefore, the cleavage without repair leads to the fragmentation of the genomic amplifications and, eventually, to the death of the cancer cells.

The term “fusion gene” as used herein means the codifying region of a gene and also, the regulatory regions and other non codifying sequences such as promoters, etc. In a preferred embodiment of the first aspect, the genomic rearrangement leads to the expression of a fusion gene selected from EWSR1-FLI1, BCR-ABL, DNAJB1-PRKACA, EML4-ALK, PAX3-FOXO1 and TPM3-NTRK1, preferably leads to the expression of fusion gene EWSR1-FLI1 or BCR-ABL. In a preferred embodiment, the cancer cells are Ewing's sarcoma cells, preferably Ewing's sarcoma cells comprising a genomic rearrangement leading to the expression of fusion gene EWSR1-FLI1. In a preferred embodiment, the cancer cells are myeloid leukaemia cells, preferably myeloid leukaemia cells comprising a genomic rearrangement leading to the expression of fusion gene BCR-ABL.

As used herein, the term “cancer inducing gene” refers to an oncogene or a proto-oncogene that has the potential to cause cancer.

In a preferred embodiment, said genomic amplifications or rearrangements which lead to the induction of the expression or to the overexpression of a cancer inducing gene are not present in non cancer cells.

In a preferred embodiment, the method comprises cleaving in at least two, three, four or five sites or even further cleavage sites, for example in the case of gene amplifications, the cleavage site may occur in as many sites as repetitions of the amplified gene are present. Therefore, the method may comprise cleaving in at least two sites to hundreds of sites in cases where the genomic rearrangement comprises hundreds of repetitions of a cancer inducing gene. In a preferred embodiment, the method comprises successive repetitions of the cleavage targeting the same or different cleaving sites. In a preferred embodiment, the method comprises cleaving in two sites and subsequently cleaving in two sites that may be the same or different. For this successive cleavage, nested gRNAs may be employed.

In a preferred embodiment, the cleavage is performed by at least one endonuclease. Said endonuclease may be a CRISPR related protein such as Cas9 or by a functional equivalent thereof, whose target site is driven by the sequence of the guide RNA. Also, the cleaving may be performed by endonucleases such as a zinc-finger nucleases (ZFN) or transcription activator-like effector nucleases (TALEN). In these cases, the target site is inherent to the nuclease and therefore at least two nucleases will be necessary to cleave the genome in at least two sites. Both of these approaches involve applying the principles of protein-DNA interactions of these domains to engineer new proteins with unique DNA-binding specificity. These methods have been widely successful for many applications.

In a preferred embodiment of the method of the first aspect, the cancer cells comprise a genomic rearrangement which leads to the expression of the rearranged gene not present in non-cancer cells. Preferably, it leads to the expression of a fusion gene not present in non-cancer cells.

In a preferred embodiment of the method of the first aspect, said method comprises cleaving the genome in two sites, or in three sites or in four sites. Preferably, it consists in cleaving the genome in two sites. Preferably, it consists in cleaving the genome in three sites. Preferably, it consists in cleaving the genome in four sites.

In a preferred embodiment of the method of the first aspect, the cancer cells comprise a genomic amplification and said method comprises cleaving the genome at least in 2 sites or at least in 10 sites or at least in 100 sites. In another preferred embodiment, the genomic amplification comprises at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100 copies of the amplified genomic region which is targeted for the cleavage, so the genome is cleaved in the same number of sites as the copy number of the targeted sequence. In a preferred embodiment of the method of the first aspect, the genomic amplification is chromosomal or extrachromosomal.

In a preferred embodiment of the method of the first aspect, the cleavage is in a genomic region other than a coding region or a regulatory region, preferably the cleavage is in an intronic region, more preferably the cleavage is in an intronic region other than the splice sites. Specifically, when the genomic rearrangement leads to a genomic amplification, the cleavage may be in an intergenic region out of coding regions or regulatory regions.

The inventors have found that the method of the present invention is specially advantageous when used to eliminate the cancer cells that comprise a genome amplification (while normal cells do not comprise said amplification), because the method of the invention leads to a DNA damage in the cancer cells that arrests the cell cycle in G2 and eventually provokes cell death of the cancer cell. This method is as effective as a radiotherapy that could be directed specifically and exclusively to cancer cells, with the enormous advantage that the side effects are minimized because the method of the invention does not affect normal cells not bearing the genome amplifications.

In a preferred embodiment of the method of the first aspect, the genomic amplification comprises at least one gene selected from MYCN, MYC, FOXO1, ERBB2(Her2), EGFR. MET, FGFR2, CCND1, MDM2, RAB25, MDM4, KRAS, AURKA, TERT and a combination thereof, preferably comprises gene MYCN or MYC.

In a preferred embodiment of the method of the first aspect, the cancer cells are neuroblastoma cells, preferably neuroblastoma cells comprising a genomic amplification comprising gene MYCN or wherein the cancer cells are medulloblastoma cells, preferably medulloblastoma cells comprising a genomic amplification comprising gene MYC.

In a preferred embodiment of the method of the first aspect, cleaving the genome leads to a genome damage, a deletion, an inversion, a frameshift or any combination thereof.

In a preferred embodiment of the method of the first aspect, at least two of the cleaving sites are in introns. The method of the present invention comprises the cleavage of the genome of the cancer cells preferably in intronic regions. These regions are eliminated during the process known as splicing. Introns are removed from primary transcripts by cleavage at conserved sequences called splice sites. These sites are found at the 5′ and 3′ ends of introns. Most commonly, the RNA sequence that is removed begins with the dinucleotide GU at its 5′ end, and ends with AG at its 3′ end. These consensus sequences are known to be critical, because changing one of the conserved nucleotides results in inhibition of splicing. The consensus sequence for an intron (in IUPAC nucleic acid notation) is: G-G-[cut]-G-U-R-A-G-U (donor site) . . . intron sequence . . . Y-U-R-A-C (branch sequence 20-50 nucleotides upstream of acceptor site) . . . Y-rich-N-C-A-G-[cut]-G (acceptor site).

The inventors have observed that upon cleaving the genome of the cancer cells in two sites, it can occur that the cleaved sequence inserts itself in the same position but in inverse orientation (an inversion), which leads to the death of the cancer cell because either the fusion protein is not produced (the inversion leads to a truncated protein) or the induction of the expression or to the overexpression of a cancer inducing gene is prevented.

The expression “genomic rearrangement” refers to a deletion, an insertion or a genomic amplification. Also, a genomic rearrangement may be a translocation of a chromosomal region, such as those that lead to the production of fusion proteins.

Preferred genomic rearrangements are listed in the following table 1.

| | Disease | Fusion Gene |
|---|---|---|
| LEUKEMIA | Acute myeloid | RUNX1-RUNX1T1 |
| | leukemia (AML) | CBFB-MYH11 |
| | | KMT2A-MLLT3 |
| | | RPN1-MECOM |
| | | DEK-NUP214 |
| | | PVT1-MECOM |
| | | RUNX1-MECOM |
| | Acute promyelocytic | PML-RARA |
| | leukemia (APL) | ZBTB16-RARA |
| | Acute lymphocytic | ETV6-RUNX1 |
| | leukemia (ALL) | BCR-ABL1 |
| | | TCF3-PBX1 |
| | | KMT2A-AFF1 |
| | | PICALM-MLLT10 |
| | | IGH-CEBPA |
| | | TCF3-HLF |
| | | TRA-MYC |
| | Chronic myeloid | BCR-ABL1 |
| | leukemia (CML) | |
| | Chronic lymphocytic | IGH-BCL1 |
| | leukemia (CLL) | IGH-BCL2 |
| | | IGH-BCL3 |
| SARCOMA/ | Ewing’s sarcoma | EWS-FLI1 |
| BONE | | EWS-ERG |
| | | EWS-ETV1 |
| | | EWS-FEV |
| | | EWS-E1AF |
| | Alveolar | PAX3/FOXO1 |
| | rhabdomyosarcoma (RMS) | PAX7-FOXO1 |
| | Congenital spindle cell RMS | VGLL2-CITED2 |
| | | VGLL2-NCOA2 |
| | | TEAD1-NCOA2 |
| | Alveolar soft-part sarcoma | ASPSCR1-TFE |
| | Extraskeletal myxoid | EWS-TEC |
| | chondrosarcoma | TAF2N-TEC |
| | Fibromyxoid sarcoma | FUS-CREB312 |
| | Endometrial stromal sarcoma | JAZF1-JJAZ1 |
| | Angiomatoid fibrous | EWSR1-CREB1 |
| | histiocytoma | FUS-ATF1 |
| | Juvenile fibrosarcoma | ETV6-NTRK3 |
| | Myxoid chondrosarcoma | EWS-NR4A3 |
| | | TFC12-NR4A3 |
| | | TAF2N-NR4A3 |
| | | SYT-SSX1 |
| | Synovial sarcoma | SYT-SSX2 |
| | | SYT-SSX4 |
| | Mixoid liposarcoma | FUS-CHOP |
| | | EWS-CHOP |
| | Spindle cell sarcoma | MLL4-GPS2 |
| | Dermatofibrosarcoma | COL1A1PDGFB |
| | protuberans (DFSP) | |
| | Clear cell sarcoma | EWS-ATF1 |
| | Soft tissue angiofibroma | AHRR-NCOA2 |
| | Undifferentiated round | BCOR-CCNB3 |
| | cell sarcoma (URCS) | CIC-DUX4L10 |
| | | CIC-DUX4 |
| | Chondroid lipoma | C11ORF95-MKL2 |
| | Mesenchymal chondrosarcoma | HEY1-NCOA2 |
| | Biphenotypic sinonasal sarcoma | PAX3-M4ML3 |
| | Despoplastic small | EWS-WT1 |
| | round cell tumor | |
| LYMPHOMAS | Follicular lymphoma | BCL2-IGH |
| | Mantle lymphoma | BCL1-IGH |
| | large cell lymphoma | NPM-ALK |
| | Burkit lymphoma | MYC-IGH |
| BRAIN | Pilocytic astrocytoma | KTAA1549-BRAF |
| TUMORS | Glioblastoma | TPM3-NTRK1 |
| | | FGFR3-TACC3 |
| | sporadic pilocytic | KIAA1549-BRA |
| | astrocytomas/some | |
| | pedriatic brain tumors | |
| | supratentorial ependymomas | C11orf95-RELA |
| | Meningioma | MN1-ETV6 |
| LIVER | fibrolamellar hepatocellular | DNAJB1_PRKACA |
| TUMORS | carcinoma | |
| KIDNEY | Clear renal cell carcinoma | SFPQ-TFE3 |
| TUMORS | | TFG-GPR1228 |
| | Mesoblastic nephroma | ETV6-NTRK3 |
| | Renal cell carcinoma | MALAT1-TFEB |
| LUNG | Lung adenocarcinoma | EML4-ALK |
| TUMORS | | LRIG3/ROS1 |
| | Non-small cell carcinoma | EML4/ALF |
| PROSTATE | Prostate | TMPRSS2-ERG |
| TUMORS | | |
| BREAST/ | Breast Cancer | BCAS4-BCAS3 |
| OVARIAN | | TEL1XR1-RGS17 |
| TUMORS | | ODZ4-NRG1 |
| | Secretory breast cancer | ETV6-NTRK3 |
| | Serous ovarian carcinoma | ESRRA-C11orf20 |
| COLON | Colorectal Cancer | PTPRK-RSPO3 |
| TUMORS | | TPM3-NTRK1 |
| | | EIF3E-RSPO2 |
| BLADDER | Bladder cancer | FGFR3-TACC3 |
| TUMORS | | |
| SALIVARY | Mucoepidermoid carcinomas | MECT1-MAML2 |
| GLAND | Adenoid cystic carcinoma | MYC-NFIB |
| TUMORS | Pleomorphic adenoma | CTNNB1-PLAG1 |
| ENDOCRINE | Papillary thyroid cancer (PTC) | ETV6-NTRK3 |
| CANCER | follicular thyroid cancer | PAX8-PPARG |
| OTHER | Aggressive midline carcinoma | BRD4-NUT |
| CANCER | Melanoma of soft parts | EWSR1-ATF1 |
| | Gastric cancer | CD33-SLC1A2 |
| | Disease | Amplified gene |
| LEUKEMIA | Acute myeloid | TRIB1 |
| | leukaemia (AML) | |
| | Acute promyelocytic | MYC |
| | leukemia (APL) | |
| SARCOMA/ | Rhabdomyosarcoma | MYC1V, FGFR1, GPC5 |
| BONE | Sarcoma | JUN, MAP3K5, YEATS4, CDK4, |
| | | DYRK2, MDM2, TERT |
| | Osteosarcoma | COPS3, MDM2 |
| | Soft tissue sarcoma | SKP2 |
| LYNPHOMAS | Diffuse large B cell | REL |
| | lymphoma | |
| | Hodgkin’s lymphoma | REL |
| BRAIN | Glioma | MDM4, EGFR, CDK4, MDM2, |
| TUMORS | | AKT3, CCND2, CDK6, MET |
| | Medulloblastoma | MYC |
| LIVER | Hepatocellular carcinoma | CHD1L |
| TUMORS | Liver | YAP1, BIRC2, TERT |
| BREAST/ | Ovarian | EIF5A2, EVI1, EMSY, ERBB2, |
| OVARIAN | | RPS6KB1, AKT2, RAB25, |
| TUMORS | | PIK3CA, TERT |
| | Breast | ERBB2, SHC1, CKS1B, RUVBL1, |
| | | C8orf4, LSM1, FGFR1, BAG4, |
| | | MTDH, MYC, EMSY, PAK1, CDK4, |
| | | MDM2, PLA2G10, STARD3, GRB7, |
| | | RPS6KB1, PPM1D, CCNE1, |
| | | YWHAB, ZNF217, AURKA, |
| | | PTK6, CCND1, NCOA3, |
| | Endometrial | ERBB2 |
| TESTICULAR/ | Testicular germ cell tumour | KIT, KRAS |
| PROSTATE | Prostate | MYC |
| BLADDER | Bladder | YWHAQ, E2F3, YWHAZ, ERRB2, |
| TUMORS | | AURKA, TERT |
| COLON | Colorectal | MYC, EGFR |
| TUMORS | | MYCN, EGFR, MET, WHSC1L1, |
| | | YWHAZ, MYC, CCND1, MDM2, |
| LUNG | Lung | BCL2L2, PAX9, NKX2-1, |
| TUMORS | | KIAA0174, DCUN1D1, EEF1A2, |
| | | MYCL1, SKP2, NKX2-8, TERT |
| RENAL TUMORS | | |
| PANCREATIC | Pancreatic | ARPC1A, SMURF1, MED29 |
| TUMORS | Pancreatobillary | GATA6 |
| OTHER | Head and neck | DCUN1D1, TERT |
| TUMORS | Malignant melanoma | MITF, CCND1, CDK4 |
| | Neuroblastoma | MDM2, MYCN |
| | Oesophageal | PRKCI, ZNF639, SKP2, EGFR, |
| | | SHH, DYRK2, ERBB2, CCNE1, |
| | | AURKA |
| | Esophageal carcinoma | ERBB2, TERT |
| | Oral squamous cell | CCND1 |
| | carcinoma | |
| | Gastric | RAB23, MET, MYC, |
| | | ERBB2, CDC6, FGFR2 |
| | Stomach | TERT |
| | Laryngeal squamous | FADD |
| | cell carcinoma | |
| | Retinoblastoma | E2F3, MDM4 |

More preferred cancers comprising genomic rearrangements are fibrolamellar hepatocellular carcinoma, non-small cell lung cancer, alveolar rhabdomyosarcoma, glioblastoma, colorectal cancer, acute lymphocytic leukemia, Ewing sarcoma, bladder cancer, neuroblastoma, medulloblastoma, breast cancer, gastric cancer, oral squamous carcinoma, osteosarcoma, ovarian cancer, retinoblastoma, testicular germ cell tumor or adrenocortical carcinoma.

Insertions may vary in size, from a few nucleotides to hundreds or more than a thousand nucleotides. Said insertions can include codifying sequences or non codifying sequences. They can also include a suicide gene, which is inserted after cleaving the genome in at least two sites. The inserted DNA can either be endogenous or exogenous.

In a preferred embodiment, when the genome is cleaved and the cleavage leads to an insertion, said insertion is the consequence of the repair of the cleavage, and not the insertion of any exogenous DNA.

In a preferred embodiment, the genomic rearrangement is other than an insertion. In a preferred embodiment, the genomic rearrangement is an insertion of a sequence other than a suicide gene.

In a preferred embodiment of the first aspect, at least two of the cleaving sites are in introns chosen in a way that the mature mRNA resulting from the fusion gene after the deletion is truncated or has a different sequence due to a frameshift. In the case of translocations that bind one promoter to the coding sequence of another gene, one of the sgRNAs will have its target domain in an intron and the other will have its cleavage site located before or after the promoter sequence but without affecting said sequence, so that the expression of the wild type gene controlled by this promoter is not altered.

The cancer cells may comprise both a genomic rearrangement that leads to a fusion gene and a genomic rearrangement that leads to the induction of the expression or to the overexpression of a cancer inducing gene, such as an oncogene. Also, the cancer cells may comprise both a genomic rearrangement that leads to a fusion gene and a genomic amplification that leads to the induction of the expression or to the overexpression of a cancer inducing gene, such as an oncogene.

The method of the invention achieves a cleavage of the genome in those at least two sites exclusively in the cancer cells because in the case of fusion genes, only the cancer cells have fusion genes, and in the case of genomic rearrangements leading to the induction of the expression or to the overexpression of a cancer inducing gene, only those cells have said genomic rearrangements. In the case of amplifications, the target domain of the gRNA (therefore the cleavage sequence) is repeated so there are at least two cleavage sites although only one gRNA is used, because the target domain is the same. The number of cleavage sites in case of amplifications will depend on the number of repetitions but only one single gRNA is necessary. Therefore, in this case the cleavage in at least two sites does not imply that the sites have different target domain sequences. Gene amplification is a copy number increase of a restricted region of a chromosome arm. The amplified copy or copies may appear on the same chromosome as the parental alleles, but may also be translocated to other chromosome(s) or even to extra-chromosomal acentric elements. Amplified DNA can be organized differently: in extrachromosomal material (double minutes, DMs), in tandem in a locus (homogeneously staining region, HSR) or distributed in several regions of the genome (interdispersed)³⁸. Some oncogene amplifications are associated with specific tumors and usually represent an indicator of poor prognosis^{32, 36} DMs are small fragments of extrachromosomal DNA, which have been observed in a large number of human tumors, DMs are composed of chromatin and replicate in the nucleus of the cell during cell division. Unlike typical chromosomes, they are composed of circular fragments of DNA, and contain no centromere or telomere. Amplified oncogenes give the cells selective advantages for growth and survival. The DNA amplification will usually lead to a corresponding increase in expression of the genes contained in the amplicon. The amplicon can be quite large (commonly the size range is 100 kb to several megabases) and contain several genes, but it is thought that one gene (usually an oncogene) is the major target of amplification, providing the cancerous cell with a growth or survival advantage when overexpressed³³. The homogeneously staining regions (HSR) just as the DMs, will contain copies of an amplified DNA segment (the amplicon), leading to cellular overexpression of the genes contained in the segment. In a single HSR there are usually many amplicon copies arranged in tandem array.

Gene amplification refers to an increase in the number of copies of the same gene rather than to an increase in its rate of transcription. It results from gene duplication that has been repeated many times over, producing from 3 (amplified) to 10 (moderately amplified) or to 100-1000 (highly amplified) copies of the gene. Examples of gene amplification are the ribosomal genes and histone genes that are found clustered in tandem (end-to-end) arrays in the genome. In actively growing or differentiating tissues such as those seen in embryonic development, ribosomal RNA is needed in large amounts that can only be provided by multiple copies of the same gene. Gene amplification is a relatively frequent event in cancer genomes. Amplification-dependent overexpression of 64 known driver oncogenes were found in 587 tumors (40%); genes frequently observed were MYC (25%) and MET (18%) in colorectal cancer; SKP2 (21%) in lung squamous cell carcinoma; HIST1H3B (19%) and MYCN (13%) in liver cancer; KIT (57%) in gastrointestinal stromal tumors; and FOXL2 (12%) in squamous cell carcinoma across tissues.

In a preferred embodiment of the first aspect, the cleavage does not result in the insertion of an exogenous gene, like a suicide gene, like the ones disclosed in WO2016094888 A1.

Another aspect of the present invention relates to a method for eliminating cancer cells, wherein said cells comprise a genomic rearrangement which leads the expression a fusion gene not present in non-cancer cells, said method comprising cleaving the expression product of said fusion gene in at least one site.

The cleavage of the mRNA of the fusion gene is specific for the cancer cells and leads to the degradation of the mRNA, preventing the translation of the fusion protein, which in turn leads to the death of the cancer cell.

In a preferred embodiment of this aspect, the cleavage is done using endonuclease Cas13. The cleavage of Cas13 of the RNA of the fusion gene is exclusive of the cancer cells and leads to the degradation of the RNA in the cell and eventually to its death. The Cas13 enzyme is a CRISPR RNA (crRNA)-guided RNA-targeting CRISPR effector²¹⁻²⁷. Under the guidance of a single crRNA, Cas13 can bind and cleave a target RNA carrying a complementary sequence. Through this mechanism, the CRISPR-Cas13 system can effectively knockdown mRNA expression in mammalian cells with an efficacy comparable with RNA interference technology and with improved specificity^{28,29}. X. Zhao and collaborators³⁰ have demonstrated that the CRISPR-Cas13 system can be engineered for the efficient and specific knockdown of mutant KRAS-G12D mRNA in pancreatic cancer models.

In a preferred embodiment of this aspect, only one gRNA is used. This gRNA has its targeting domain in the expression product of the fusion gene. Preferred gRNAs are those codified by sequences SEQ ID NO: 135, SEQ ID NO: 136, SEQ ID NO: 137 and SEQ ID NO: 138, useful for cleaving the expression products of fusion genes DNAJB1-PRKACA, EML4-ALK, PAX3-FOXO1 and TPM3-NTRK1, respectively.

A second aspect relates to a kit of parts comprising an endonuclease, preferably selected from a zinc-finger nuclease (ZFN) and a transcription activator-like effector nuclease (TALEN), wherein said endonuclease specifically cleaves the genome in a genomic region other than a coding region or a regulatory region, preferably in an intronic region of a genomic amplification, more preferably the cleavage is in an intronic region other than the splice sites. Said kit of parts may comprise the endonuclease or a sequence coding said endonuclease, preferably in an expression vector.

A third aspect relates to a kit of parts comprising an endonuclease capable of cleaving a messenger RNA (mRNA), such as the CRISPR associated protein Cas13 or another endonuclease derived from said Cas13 or a functional equivalent thereof (or a sequence coding said endonuclease); and at least one gRNA, preferably one gRNA, with its targeting domain in the expression product of a fusion gene present in cancer cells and absent in non-cancer cells. In a preferred embodiment, the kit comprises the nuclease of SEQ ID NO: 126 or a functional equivalent thereof and a gRNA selected from SEQ ID NO: 144, SEQ ID NO: 145, SEQ ID NO: 146 and SEQ ID NO: 147. In a preferred embodiment, the kit consists essentially of the nuclease of SEQ ID NO: 126 or a functional equivalent thereof and a gRNA selected from SEQ ID NO: 144, SEQ ID NO: 145, SEQ ID NO: 146 and SEQ ID NO: 147. Preferably, the sequence that codifies for the nuclease of SEQ ID NO: 126 is SEQ ID NO: 127.

A fourth aspect relates to a nucleic acid codifying for a nuclease capable of cleaving a messenger RNA (mRNA), such as the CRISPR associated protein Cas13 or another endonuclease derived from said Cas13 or a functional equivalent thereof; and at least one gRNA, preferably one gRNA, with its targeting domain in the expression product of a fusion gene present in cancer cells and absent in non-cancer cells. In a preferred embodiment, the nucleic acid codifies for the nuclease of SEQ ID NO: 126 or a functional equivalent thereof and for a gRNA selected from SEQ ID NO: 144, SEQ ID NO: 145, SEQ ID NO: 146 and SEQ ID NO: 147.

A fifth aspect relates to the use of the above mentioned methods, kits or nucleic acids for the treatment of cancer, preferably for the treatment of fibrolamellar hepatocellular carcinoma, non-small cell lung cancer, alveolar rhabdomyosarcoma, glioblastoma, colorectal cancer, acute lymphocytic leukemia, Ewing sarcoma, bladder cancer, neuroblastoma, medulloblastoma, breast cancer, gastric cancer, oral squamous carcinoma, osteosarcoma, ovarian cancer, retinoblastoma, testicular germ cell tumor or adrenocortical carcinoma.

In a preferred embodiment of the method of the first aspect, the cleaving is done by an endonuclease selected from a CRISPR associated protein, a zinc-finger nuclease (ZFN) and a transcription activator-like effector nuclease (TALEN). Preferably, the cleaving is done by a Cas protein, preferably Cas9 or a functional equivalent thereof. In a preferred embodiment, the target of said endonuclease is in an intron of a fusion gene present in cancer cells and absent in non-cancer cells and wherein said target is not patient-specific. The target of said endonuclease may be in an intron or an exon or a noncoding sequence including promoter and 5′ and 3′ ends. In a preferred embodiment, the target of said endonuclease is not in a coding sequence or in a regulatory sequence. In a preferred embodiment, the target of the endonuclease or endonucleases is not in an exon or a non-coding sequence that is including a promoter, an enhancer or any other regulatory sequence. In a preferred embodiment, the target of said endonuclease is in an intron. In a more preferred embodiment, the target is in an intron sequence other than the splice sites. For example, the target for the cleavage may be in intergenic sequences, especially in the case of rearrangements leading to genomic amplifications.

In a preferred embodiment of the method of the first aspect, at least two guide RNAs are used to target the cleaving of the genome.

A second aspect of the present invention related to a kit of parts comprising at least two endonucleases, preferably selected from a zinc-finger nuclease (ZFN) and a transcription activator-like effector nuclease (TALEN), wherein said endonucleases specifically cleave the genome in at least two sites (each endonuclease cleaves in one specific site) and wherein said cleavages lead to either a deletion, a frameshift and/or an insertion in the genome, preferably a deletion and/or a frameshift.

In a preferred embodiment, the kit of parts comprises an endonuclease, preferably selected from a zinc-finger nuclease (ZFN) and a transcription activator-like effector nuclease (TALEN), wherein said endonuclease specifically cleaves the genome in an intronic region of a genomic amplification, preferably the cleavage is in an intronic region other than the splice sites.

In a preferred embodiment, the kit of parts comprises at least two ZFNs or a nucleic acid encoding at least two ZFNs. In another preferred embodiment, the kit of parts comprises at least two TALENs or a nucleic acid encoding at least two TALENs. In another preferred embodiment, the kit of parts comprises at least one ZFN and at least one TALEN or a nucleic acid encoding at least one ZFN and at least one TALEN.

In a preferred embodiment, the kit of parts comprises: (a) a CRISPR associated endonuclease, preferably a Cas protein, more preferably Cas9 or Cas13, even more preferably Cas9 or a functional equivalent thereof; and (b) at least one gRNA is used to target the cleaving of the genome, preferably at least two gRNAs are used to target the cleaving of the genome. In a preferred embodiment, the kit of parts comprises (a) a CRISPR associated endonuclease, preferably a Cas protein, more preferably Cas9 or Cas13 or a functional equivalent thereof, even more preferably Cas9; and (b) at least a pair of gRNAs that have a targeting domain in a genomic rearrangement present in a cancer cell which leads either to the expression a fusion gene not present in non-cancer cells, or to genomic amplifications or rearrangements which lead to the induction of the expression or to the overexpression of a cancer inducing gene. In a preferred embodiment, the kit of parts consists of a CRISPR associated endonuclease, preferably a Cas protein, more preferably Cas9 or Cas13 or a functional equivalent thereof, even more preferably Cas9; and at least one gRNA, preferably a pair of gRNAs that have a targeting domain in a genomic rearrangement present in a cancer cell which leads either to the expression a fusion gene not present in non-cancer cells, or to genomic amplifications or rearrangements which lead to the induction of the expression or to the overexpression of a cancer inducing gene. As used herein, the term “guide RNA” and “single guide RNA” are used interchangeably and are abbreviated as “gRNA” and “sgRNA”.

A preferred embodiment of the kit of parts of the present invention comprises: (a) the nuclease with amino acid sequence SEQ ID NO: 1; and (b) the pair of gRNAs with nucleotide sequences SEQ ID NO: 2 and SEQ ID NO: 3; or the pair of gRNAs with nucleotide sequences SEQ ID NO: 4 and SEQ ID NO: 5; or a pair of gRNAs with nucleotide sequences SEQ ID NO: 128 or SEQ ID NO: 129 and SEQ ID NO: 130 or SEQ ID NO: 131; or a pair of gRNAs with nucleotide sequences SEQ ID NO: 132 or SEQ ID NO: 133 and SEQ ID NO: 134 or SEQ ID NO: 135; or a pair of gRNAs with nucleotide sequences SEQ ID NO: 136 or SEQ ID NO: 137 and SEQ ID NO: 138 or SEQ ID NO: 139; or a pair of gRNAs with nucleotide sequences SEQ ID NO: 140 or SEQ ID NO: 141 and SEQ ID NO: 142 or SEQ ID NO: 143.

A preferred embodiment of the kit of parts of the present invention comprises: (a) the nuclease with amino acid sequence SEQ ID NO: 1; and (b) at least one gRNA with nucleotide sequence SEQ ID NO: 148 or SEQ ID NO: 149 or both. Another preferred embodiment of the kit of parts of the present invention comprises: (a) the nuclease with amino acid sequence SEQ ID NO: 1; and (b) at least one gRNA with nucleotide sequence SEQ ID NO: 148 or SEQ ID NO: 149 or SEQ ID NO: 152 or SEQ ID NO: 153 or a combination thereof.

Another aspect of the present invention relates to a nucleic acid comprising the codifying sequence for (a) a CRISPR associated endonuclease, preferably a Cas protein, more preferably Cas9 or Cas13 or a functional equivalent thereof, even more preferably Cas9; and (b) at least one gRNA that has a targeting domain in the expression product of a fusion gene or at least a pair of gRNAs that have a targeting domain in a genomic rearrangement present in a cancer cell which leads either to the expression of a fusion gene not present in non-cancer cells, or to genomic amplifications or rearrangements which lead to the induction of the expression or to the overexpression of a cancer inducing gene.

In an embodiment of this aspect, the at least one gRNA has a targeting domain in a genomic amplification present in a cancer cell and absent in non-cancer cells, preferably in a genomic region other than a coding region or a regulatory region, more preferably in an intronic region of said genomic amplification, more preferably in an intronic region of an oncogene other than the splice sites.

Another aspect of the present invention relates to a nucleic acid comprising essentially the codifying sequence for (a) a CRISPR associated endonuclease, preferably a Cas protein, more preferably Cas9 or Cas13 or a functional equivalent thereof, even more preferably Cas9; and (b) at least one gRNA that has a targeting domain in the expression product of a fusion gene or at least a pair of gRNAs that have a targeting domain in a genomic rearrangement present in a cancer cell which leads either to the expression of a fusion gene not present in non-cancer cells, or to genomic amplifications or rearrangements which lead to the induction of the expression or to the overexpression of a cancer inducing gene. Said nucleic acid may comprise other elements, such as promoters, enhancers, etc. well known to the skilled person and which allow the expression of the endonuclesase and the gRNAs in the target cancer cells.

A particularly preferred embodiment of the present invention relates to a nucleic acid comprising the codifying sequence for: (a) the nuclease with amino acid sequence SEQ ID NO: 1, preferably the codifying sequence SEQ ID NO: 32, and (b) the pair of gRNAs with nucleotide sequences SEQ ID NO: 2 and SEQ ID NO: 3; or the pair of gRNAs with nucleotide sequences SEQ ID NO: 4 and SEQ ID NO: 5; or a pair of gRNAs with nucleotide sequences SEQ ID NO: 128 or SEQ ID NO: 129 and SEQ ID NO: 130 or SEQ ID NO: 131; or a pair of gRNAs with nucleotide sequences SEQ ID NO: 132 or SEQ ID NO: 133 and SEQ ID NO: 134 or SEQ ID NO: 135; or a pair of gRNAs with nucleotide sequences SEQ ID NO: 136 or SEQ ID NO: 137 and SEQ ID NO: 138 or SEQ ID NO: 139; or a pair of gRNAs with nucleotide sequences SEQ ID NO: 140 or SEQ ID NO: 141 and SEQ ID NO: 142 or SEQ ID NO: 143.

Preferred pairs of gRNAs are listed below for four preferred cancers (two gRNAs are provided for each cleavage site for each disease):

Fibrolamellar Hepatocellular Carcinoma.

Fusion gene DNAJB1-PRKACA
> DNAJB1
SEQ ID NO: 128:
Position 11252; Strand: 1; Sequence:
GATGTCGCGTGTCGCTGAAA; PAM: GGG;
Specificity Score: 98.2633296; Efficiency Score:
46.297448948149196
SEQ ID NO: 129:
Position 12068; Strand: 1; Sequence:
CAGGAGCCGACCCCGTTCGT; PAM: GGG;
Specificity Score: 95.6957217; Efficiency Score:
54.78256070394193
> PRKACA
SEQ ID NO: 130:
Position: 1935; Strand: 1; Sequence:
GTCGGAACTATTGGTCGAAA; PAM: AGG;
Specificity Score: 94.8121995; Efficiency Score:
49.440679428197285
SEQ ID NO: 131:
Position: 846; Strand: 1; Sequence:
CATGGCACGTATGACCGCTG; PAM: GGG;
Specificity Score: 91.353957; Efficiency Score:
69.65437430185875
Non-small cell lung cancer. Fusion gene EML4-ALK
> EML4
SEQ ID NO: 132:
Position: 42262641; Strand: 1; Sequence:
ACTTATAAGTATAGGGAATC; PAM: AGG;
Specificity Score: 73.9472732; Efficiency Score:
41.01080196055957
SEQ ID NO: 133:
Position: 42263040; Strand: −1; Sequence
GGATTAGTTGAAAGACTGCC:; PAM: TGG;
Specificity Score: 71.6133454; Efficiency Score:
42.03495303486019
> ALK
SEQ ID NO: 134:
Position: 698882; Strand: 1; Sequence:
GTCCACTAAATGTGACGCCC; PAM: AGG;
Specificity Score: 92.5531067; Efficiency Score:
54.886608266105895
SEQ ID NO: 135:
Position: 698375; Strand: 1; Sequence:
GAGGACAAGCCTTGACATTC; PAM: AGG;
Specificity Score: 73.6854929; Efficiency Score:
31.53386778402246
Alveolar Rhabdomyosarcoma. Fusion gene PAX3-FOXO1
> PAX3
SEQ ID NO: 136:
Position: 96631; Strand: 1; Sequence:
TGCAGTCAGATGTTATCGTC; PAM: GGG;
Specificity Score: 92.4221555; Efficiency Score:
51.18110845580498
SEQ ID NO: 137:
D Position: 95396; Strand: 1; Sequence:
TACTGGAACTCCTAGATCCG; PAM: AGG;
Specificity Score: 87.0387327; Efficiency Score:
65.91762085633988
> FOXO1
SEQ ID NO: 138:
Position: 108815; Strand: 1; Sequence:
CAATGGTCCTTTGTCAAACG; PAM: AGG;
Specificity Score: 83.6621655; Efficiency Score:
62.541738049196596
SEQ ID NO: 139:
Position: 108095; Strand: −1; Sequence:
TGGCAACGTGAACAGGTCCA; PAM: AGG;
Specificity Score: 76.5677517; Efficiency Score:
64.81592708499454
Glioblastoma. Fusion gene TPM3-NTRK1
> TPM3
SEQ ID NO: 140:
Position: 23359; Strand: −1; Sequence:
AACCTGAATACATGGTAAGG; PAM: AGG;
Specificity Score: 71.2689516; Efficiency Score:
62.792710664503396
SEQ ID NO: 141:
Position: 23653; Strand: 1; Sequence:
TACTCTTGCTCATCAAGCAG; PAM: GGG;
Specificity Score: 69.2115539; Efficiency Score:
60.98480800289473
> NTRK1
SEQ ID NO: 142:
Position: 156877732; Strand: 1; Sequence:
CTGGATGAGCAAGCGCTGTA; PAM: TGG;
Specificity Score: 90.0534004; Efficiency Score:
47.93006273145852
SEQ ID NO: 143:
Position: 156877536; Strand: −1; Sequence:
TCAGAGAAGGACTAGACCGA; PAM:
GGG; Specificity Score: 86.3585081; Efficiency
Score: 68.2983905554927

Preferred gRNAs are listed below for several preferred cancers associated with genomic amplifications comprising at least an oncogene:

Neuroblastoma:
Gene MYCN:
gRNA:
(SEQ ID NO: 148)
CTGTCGTAGACAGCTTGTAC
Gene MYCN:
gRNA:
(SEQ ID NO: 149)
CGGTCGCAATCTGGGTCACG
Medulloblastoma:
Gene MYCN:
gRNA:
(SEQ ID NO: 148)
CTGTCGTAGACAGCTTGTAC
Gene MYCN:
gRNA:
(SEQ ID NO: 149)
CGGTCGCAATCTGGGTCACG
Gene MYC:
gRNA:
(SEQ ID NO: 152)
CATCTCCGTATTGAGTGCGA
Gene MYC:
gRNA:
(SEQ ID NO: 153)
CCCGTTAACATTTTAATTGC
Rhabdomyosarcoma:
Gene FOXO1:
gRNA:
(SEQ ID NO: 154)
ACTGTATAGCTGTACTCGGG
Colon cancer:
Gene MYC:
gRNA:
(SEQ ID NO: 152)
CATCTCCGTATTGAGTGCGA
Breast cancer:
Gene ERBB2 (Her2):
gRNA:
(SEQ ID NO: 155)
GTGGAATGCAGGTGTCATAC
Glioblastoma:
Gene EGFR:
gRNA:
(SEQ ID NO: 156)
CATGTTGGTACATCCATCCG
Lung cancer:
Gene MET:
gRNA:
(SEQ ID NO: 157)
GTTGCCGGTATAAGAGACAG
Gastric cancer:
Gene FGFR2:
gRNA:
(SEQ ID NO: 158)
GACGCAAGCATTAAACCGGG
Oral squamous carcinoma:
Gene CCND1:
gRNA:
(SEQ ID NO: 159)
CTGGGTAAAGGGTCGCCCGA
Osteosarcoma:
Gene MDM2:
gRNA:
(SEQ ID NO: 160)
CGGACCGATCACCTGAGATG
Ovarian cancer:
Gene RAB25:
gRNA:
(SEQ ID NO: 161)
GCCCTAGCGTCATACCACAA
Retinoblastoma:
Gene MDM4:
gRNA:
(SEQ ID NO: 162)
GCACTTACTCAACGGTCTCG
Testicular germ cell tumour:
Gene KRAS:
gRNA:
(SEQ ID NO: 163)
TACTAGCCTAGGAAATACTG
Bladder cancer:
Gene AURKA:
gRNA:
(SEQ ID NO: 164)
CGTACGGAGAACTTGCAGCT
Adrenocortical carcinoma:
Gene TERT:
gRNA:
(SEQ ID NO: 165)
GACGCTTATCTGACTCGGCG

Another aspect of the present invention is a nucleic acid comprising the codifying sequence for:

a. the nuclease with amino acid sequence SEQ ID NO: 1; and

b. at least one gRNA with nucleotide sequence SEQ ID NO: 148 or SEQ ID NO: 149 or both.

Another aspect of the present invention is a nucleic acid comprising the codifying sequence for:

a. the nuclease with amino acid sequence SEQ ID NO: 1; and

b. at least one gRNA with nucleotide sequence SEQ ID NO: 148 or SEQ ID NO: 149 or SEQ ID NO: 152 or SEQ ID NO: 153 or a combination thereof.

Another aspect of the present invention is a nucleic acid comprising the codifying sequence for:

a. the nuclease with amino acid sequence SEQ ID NO: 1; and

b. at least one gRNA with nucleotide sequence SEQ ID NO: 152 or SEQ ID NO: 154, or SEQ ID NO: 155, or SEQ ID NO: 156, or SEQ ID NO: 157, or SEQ ID NO: 158, or SEQ ID NO: 159, or SEQ ID NO: 160, or SEQ ID NO: 161, or SEQ ID NO: 162, or SEQ ID NO: 163, or SEQ ID NO: 164, or SEQ ID NO: 165.

Another aspect of the present invention relates to the use of any one of the methods of the invention, or of any one of the kits of parts of the invention or the nucleic acids of the invention for the treatment of cancer, preferably for the treatment of fibrolamellar hepatocellular carcinoma, non-small cell lung cancer, alveolar rhabdomyosarcoma, glioblastoma, colorectal cancer, acute lymphocytic leukemia, Ewing sarcoma, bladder cancer, neuroblastoma, medulloblastoma, breast cancer, gastric cancer, oral squamous carcinoma, osteosarcoma, ovarian cancer, retinoblastoma, testicular germ cell tumor or adrenocortical carcinoma. Preferably, for the treatment of cancers where there is a genomic rearrangement present in a cancer cell which leads either to the expression a fusion gene not present in non-cancer cells, or to genomic amplifications or rearrangements which lead to the induction of the expression or to the overexpression of a cancer inducing gene. More preferably, for the treatment of cancers where there is a fusion gene and a fusion protein specifically in cancer cells, not present in non-cancer cells. Even more preferably, for the treatment of the cancers listed in table 1. Preferably, the kit of parts of the present invention is delivered to the patient in need of the treatment by specific delivery systems that are known to be useful in each particular cancer type. Delivery systems such as viral vectors, adenoviral vectors, lentiviral vectors, AAVs and other delivery systems such as nanoparticles and macrocomplexes can be used. The administration of the kit of parts of the present invention can be through different ways, depending on the target tissue or cancer cell in the patient. Thus, the administration may be oral or parenteral, subcutaneous, intramuscular or intravenous, as well as intrathecal, intracranial, etc., depending on the patient needs.

### DESCRIPTION OF THE DRAWINGS

FIG. 1: a. Schematic representation of the type 1 (EWSR1 exon 7 fused to FLI1 exon 6) and type 2 (EWSR1 exon 7 fused to FLI1 exon 5) EWSR1-FLI1 fusion gene loci. Indicated are the sgRNAs targeting introns 3 of EWSR1 and 8 of FLI1 genes used to edit the fusion gene. b. Schematic representation of the truncated DNA generated by Cas9 edition. c. Gene editing effect on the EWSR1 and FLI1 WT intronic on-target sites: a. Schematic representation of EWSR1 and FLI1 WT genes. Indicated are the sgRNAs targeting both genes. d. Schematic representation of the indels removal by the splicing machinery. Schematic illustration of the pLVX-U6E3 0.2-H1F8.2-Cas9-2A-eGFP vector.

FIG. 2: a. EWSR1-FLI1 chimeric protein and truncated protein generated by genome editing. b Amino acid sequence of the EWSR1-FLI1 protein (Type 1). Residues corresponding to EWSR1 or to FLI1 are shown in black or grey, respectively. c. Aminoacid sequence of the edited EWSR1-FLI1 truncated protein. Deleted residues are shown crossed out, the new residues generated by the change of reading frame after the mutation are double underlined. The premature STOP codon is shown with an asterisk.

FIG. 3. Analysis of EWSR1-FLI1 DNA a. Agarose gel electrophoresis showing the results of genomic PCR analysis of edited and control A673 ES cell line using oligos flanking the DNA loci targeted by sgE3.2 and sgF8.2. The 300 bp PCR fragment denote deletion of the DNA fragment between the loci targeted by sgEW3.2 and sgFLI8. PCR analysis was done using DNA from cell cultures on days 2, 4 and 6 post-transduction (pt). Albumin is used as an internal control of the PCR reaction. b. Sanger sequencing analysis of the PCR bands. A representative deleted sequence and chromatogram are shown.

FIG. 4. Analysis of the EWSR1-FLI1 RNA. a. Agarose gel electrophoresis of the EWSR1-FLI1 RT-PCR products obtained from edited and control A673 ES cells. RT-PCR analysis was done using RNA from cell cultures on days 2, 4 and 6 pt. Arrows depict the sizes of wild type (961 bp) and deleted (150 bp) RT-PCR products. GAPDH is used as an internal control of the RT-PCR reaction b. Representative deleted cDNA sequence obtained by Sanger and chromatogram.

FIG. 5. Analysis of the EWSR1-FLI1 protein. EWSR1-FLI1 protein expression in A673 ES cells by western blot analysis. Western blot analysis was done using protein from cell cultures on days 3, 6 and 10 pt. GAPDH is used as an internal control of the assay.

FIG. 6. Proliferation and tumorigenicity in vitro assays a. Growth rate assay curves of A673 and RD-ES ES and U2OS osteosarcoma (EWSR1-FLI1) experimental and control cells. b. Colony formation assay. Representative images of wells after 2% crystal violet staining are shown of the A673, RD-ES and U2OS experimental and control cells. Graphical representation of the number of colonies formed in the A673, RD-ES and U2OS colony formation assays. p values are represented (**p<0.005; ***p<0.0005).

FIG. 7. Apoptosis in vitro assays. a. The DNA profile was analysed using propidium iodide staining and flow cytometry. The percentage of cellular apoptosis is calculated using the percentage of the Sub-G1 peak. Black plot represent A673 control cells and grey plot represent experimental EWSR1-FLI1 deleted A673 cells. b. The number of apoptotic cells was analysed using Caspase 3 immunostaining. The percentage of cellular apoptosis is calculated using the percentage of Caspase3 positive cells per field analysed. Black dots represent A673 control cells and grey dots represent experimental EWSR1-FLI1 deleted A673 cells.

FIG. 8. Genome editing specificity analysis. a. Representative G-banded methaphase with normal karyotype of WT human mesenchymal stem cells (hMSC) transduced with sgE3.2 and sgF8.2. b. FISH analysis of EWSR1 gene status. The schematic representation shows the structure and principle of the EWSR1 break-apart fluorescent in situ hybridization (FISH) probe (Kreatech KBI-10750). A break is defined when a fusion signals splits into separate signals. Co-localized fusion signals identify the normal chromosome(s) 22. Representative FISH images of control and experimental hMSCs. c. Profile plot result of a high-density array comparative genomic hybridization (aCGH) analysis covering the whole genome showing no copy number variations (CNVs) in hMSCs transduced with sgE3.2 and sgF8.2.

FIG. 9: Ex-vivo lentiviral EWSR1-FLI1 gene edition. a. Diagram showing the lentiviral vector and the approach for the ex-vivo treatment. A673 ES cells are transduced with of the pLVX-U6E3.2-H1F8.2-Cas9-2A-eGFP or control vectors and transplanted into immunocompromided mice. The xenografted mice are then observed and measured and sacrificed 30 days post cell injection. b. Tumour growth (mm3) over the 28 days following subcutaneous cell injection. The plot shows medians and ranges; p values are represented (*p<0.05, **p<0.005). c. Mice were sacrificed after 30 days and their tumours collected. Pictures show representative tumours of control and experimental mice. D. Representative images of Ki-67 proliferation marker and Caspase3 apoptosis marker immunostaining assays on A673 ES experimental and control cells. e. Survival curve comparing mice injected with experimental or control A673 cells.

FIG. 10: In-vivo adenoviral EWSR1-FLI1 gene edition. a. Diagram showing the control Cas9 and sgE3.2, sgF8.2 and Cas9 adenoviral vectors and schedule used for the in-vivo gene edition assays. A673 ES cells are injected in the flanks of immunocompromised mice, when reached a defined size the adenoviral, control vector and PBS are injected 4 times (every 3 days) in the xenografted tumours. The xenografted mice are then observed and measured and sacrificed 30 days post cell injection. b. Tumour growth (mm3) over the 23 days. The plot shows medians and ranges; p values are represented (*p<0.05, **p<0.005). Mice were sacrificed after 25 days and their tumours collected. Pictures show representative tumours of control and experimental mice. c. Representative images of Cas9 immunostaining assays on A673 Ewing sarcoma experimental and control cells. d. Representative images of Ki-67 proliferation and Caspase3 apoptosis markers immunostaining assays on A673 Ewing sarcoma experimental and control cells e. Survival curve comparing mice treated with sgRNAs-Cas9 experimental adenoviral vector, Cas9 control adenoviral vector or PBS.

FIG. 11: a. Schematic representation of the BCR-ABL1 fusion gene. Indicated are the sgRNAs targeting introns 8 of BCR and 1 of ABL1 genes used to edit the fusion gene. b. Schematic representation of the BCR-ABL1 chimeric and truncated protein generated by Cas9 edition. b. Amino acid sequence of the BCR-ABL protein (p210). Residues corresponding to BCR or to ABL are shown in black or grey, respectively. ABL DNA binding domain is underlined. Amino acid sequence of the edited BCR-ABL truncated protein. Deleted residues are shown crossed out, the new residues generated by the change of reading frame after the mutation are shown in italics. The premature STOP codon is shown with an asterisk. Analysis of BCR-ABL1 RNA, and in vitro viability. Four pairs (BA1, BA2, BA3 and BA4) of sgRNAs targeting both BCR and ABL1 introns were tested. b. Agarose gel electrophoresis showing the BCR-ABL1 RT-PCR products obtained from experimental and control K562 Chronic Myeloid Leukaemia cell line. RT-PCR analysis was done using RNA from cell cultures on day 2 post-nucleofection. Arrows depict the sizes of wild type (1125 bp) and deleted (458 bp) RT-PCR products. GAPDH is used as an internal control of the RT-PCR reaction. A representative deleted cDNA sequence obtained by Sanger and chromatogram is shown at the bottom. c. Colony formation assay. Representative images of wells after 2% crystal violet staining are shown of the K562 experimental and control cells. Graphical representation of the number of colonies formed in the K562 colony formation assays. p values are represented (***p<0.0005). d. Apoptosis in vitro assays. The DNA profile was analysed using propidium iodide staining and flow cytometry. The percentage of cellular apoptosis is calculated using the percentage of the Sub-G1 peak. Black plot represent K563 control cells and grey plots represent experimental BA1, BA2, BA3 and BA4 edited K562 cells.

FIG. 12. In-vivo adenoviral BCR-ABL1 gene edition. a. Tumour growth (mm3) over the 30 days following subcutaneous cell injection and 4 in-vivo adenoviral treatments (every 3 days). The plot shows medians and ranges; p values are represented (**p<0.05). b. Mice were sacrificed after 30 days and their tumours collected. Pictures show representative tumours of control and experimental mice. c. Survival curve comparing mice treated with sgRNAs-Cas9 experimental and control adenoviral vectors.

FIG. 13. Strategy representing intronic CRISPR-mediated targeting of amplified genes illustrating the genomic structure in normal cells and cells with amplifications.

FIG. 14. Representative FISH images. MYCN was used as a probe. SKNAS present two copies of MYCN. IMR32 shows a HSR amplification. LANS presents a double minutes amplification.

FIG. 15. CRISPR-mediated targeting of MYCN intron inhibits in vitro cell growth. Growth rate assay curves of IMR32, LANS and SKNAS edited (sgMYCN), transfected with a non-targeting sgRNA (sgNT) or wild-type (WT) cells. (n=3). RT-PCR products from edited and control IMR32 cells. The analysis was done using extracted RNA of cells at day 3 pt. GAPDH was used as an internal control of the RT-PCR reaction.

FIG. 16. Statistical analysis of the number of colonies of SKNAS control cell line (A), IMR32 (B) and LANS (C) (n=3).

FIG. 17. A. DNA profile analysis by propidium iodide staining and flow cytometry in SKNAS, IMR32 and LANS cells lines, treated (sgMYCN) or controls (WT and sgNT). B. Graphical representations of the G2 analysis. (n=3).

FIG. 18. Representative immunofluorescence images of SKNAS, IMR32 and LANS transfected with constructs encoding sgMYCN stained with anti-H2AX antibody to visualize DNA damage foci. DNA was counterstained with DAPI.

FIG. 19. Growth rate assay curves of MDB-HTB-185 medulloblastoma cell line transfected with two sgRNAs targeting MYC gene (sgMYC-1 and sgMYC-2), a non-targeting sgRNAs (sgNT) or wild-type (control) cells. (n=3).

### EXAMPLES

Precise Deletion of Fusion Genes Via CRISPR-Cas9.

To elucidate whether the fusion gene deletion (or rearrangement) strategy is a good gene therapy approach to treat cancer, the inventors have chosen as test models two well characterized fusion genes representative of the two major classes of clinically relevant transcription fusions: EWSR1-FLI1 Ewing's sarcoma (ES) transcription factor and BCR-ABL chronic myeloid leukaemia (CIVIL) tyrosine kinase fusion genes.

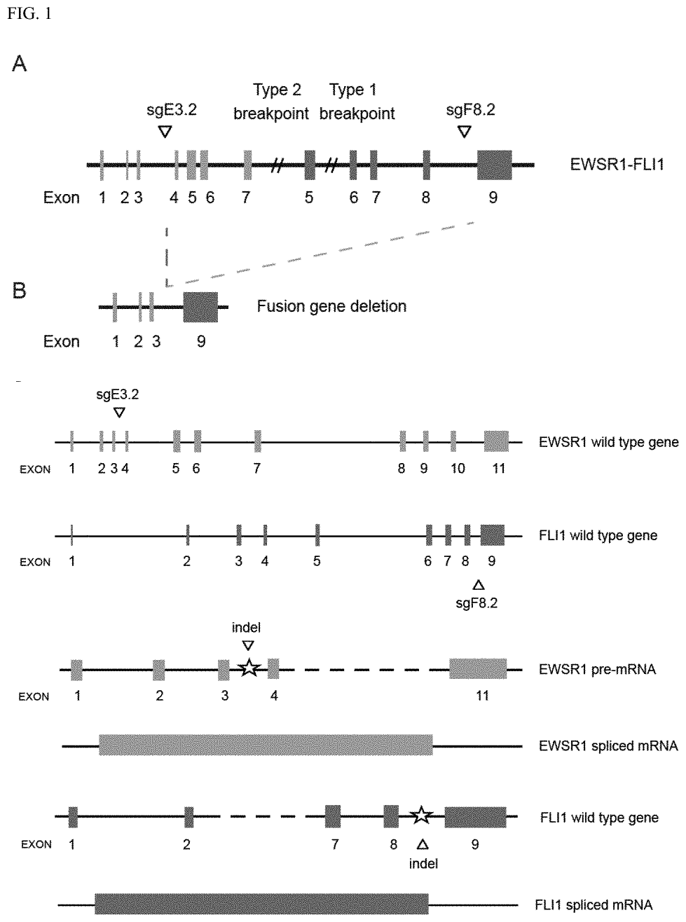

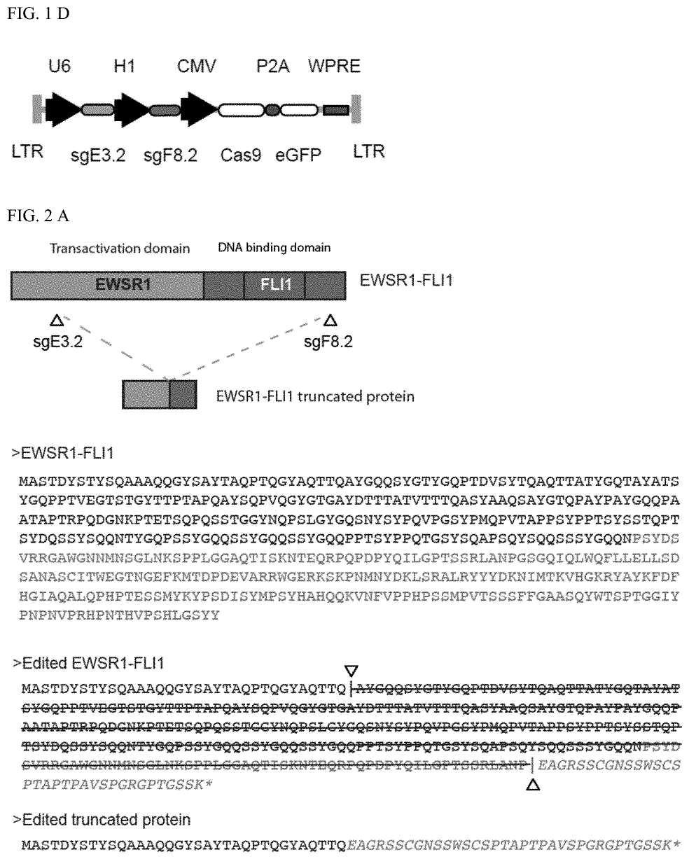

Ewing's sarcoma (ES), the second most common cancer involving bone in children, is characterized by a chromosomal translocation that fuses the strong transactivation domain of the RNA binding protein EWSR1, with the DNA binding domain of an ETS protein, most commonly FLI1. EWSR1-FLI1 acts as a transcriptional factor, and numerous studies have demonstrated a strict dependency on EWSR1-FLI1 expression of ES cells. Two main EWSR1-FLI1 subtypes have been described, fusing the EWSR1 exon 7 to FLI1 exon 6 (so-called type 1) or to FLI1 exon 5 (so-called type 2 (FIG. 1a). Two ES cell lines (A673 and RD-ES) harbouring two different isoforms, type 1 and type2, of the EWSR1-FLI1 fusion gene have been chosen as model systems.

ES and CIVIL have been selected as test models, but it is important to keep in mind that the overall approach is potentially applicable to all neoplasias addicted to the expression of fusion genes or the enforced expression of oncogenes produce by chromosomal rearrangements whose removal or modification via genome editing would cause death of tumour cells.

Selection of sgRNAs that Target EWSR1-FLI1 Fusion Gene

To selectively target both isoforms of EWSR1-FLI1 with the same CRISPR tool, a pair of sgRNAs targeting introns 3 of EWSR1 and intron 8 of FLI1 was designed (FIG. 1a and Table 2). The targeted introns were selected first, to generate a large deletion including key functional domains of the fusion gene, secondly to induce a frameshift of the remaining 3′ region of the FLI1 gene, and thirdly to include all the hotspot introns harbouring breakpoints in human patients. The design of the sgRNAs in intronic regions guaranteed no modification of the wild type EWSR1 and FLI1 proteins in non-tumour cells because any indel generated by the NHEJ repair of the on-target introns in wild type (WT) genes will be removed in the mRNA. This is because the splicing machinery will remove any indel generated in the on-target sites of both sgRNAs in the introns of wild type genes after nonhomologous end-joining (NHEJ) repair of the double strand breaks (DSBs) generated by the Cas9 nuclease (FIG. 1c). Consequently, deletions will only take place in those cells harbouring the fusion gene with both on-target intronic regions in the same chromosome (FIG. 1a).

TABLE 2
sgRNA sequences used for CRISPR-based gene editing Oligonucleotide sequences
used for PCR, RT-PCR and NGS analysis.
sgRAs

| | | | | | |
|---|---|---|---|---|---|
| sgE3.1 | SEQ ID | TAAGAGGACATACAGCGTTC TGG | sgF6.1 | SEQ ID | ATTTGGACCTGTGGCGATAT GGG |
| | NO: 33 | | | NO: 34 | |
| sgE3.2 | SEQ ID | TGGTTGCACAGTAAGTGGCG GGG | sgF6.2 | SEQ ID | AAGACGTCTTGCTCCCCTCG GGG |
| | NO: 2 | | | NO: 51 | |
| sgE6.1 | SEQ ID | CGGGCGGATCATATAAGGTC AGG | sgF8.1 | SEQ ID | CAAGTCGATCCCAATGTCGA AGG |
| | NO: 35 | | | NO: 36 | |
| sgE6.2 | SEQ ID | TAGATCGCGTACTCCATCCT GGG | sgF8.2 | SEQ ID | AGTGGGCCACACTGCGACAA GGG |
| | NO: 37 | | | NO: 3 | |
| sgB1 | SEQ ID | TATCCGAGGCACGTTAAGGG | sgA1 | SEQ ID | CACGAGGTTGACGCACCAGA |
| | NO: 4 | | | NO: 5 | |
| sgB2 | SEQ ID | GACATGACCATGGGTAAGCG | sgA2 | SEQ ID | TCCCTAATAGTGATGGCGCT |
| | NO: 38 | | | NO: 39 | |

PCR/RT-PCR EF deletion detection primers

| | | | | | |
|---|---|---|---|---|---|
| Ex3 EWSR1 | SEQ ID | GCCCAGCCCACTCAAGGATA | Ex9 FLI1 rv | SEQ ID | TTGGGGTTGGGGTAGATTCC |
| fw | NO: 40 | | | NO: 41 | |
| qEWSF1 | SEQ ID | GCAGGGCTACAGTGCTTAC | qEWSFLI rv | SEQ ID | GCAGCTCCAGGAGGAATTG |
| fw | NO: 42 | | | NO: 43 | |

On target detection primers

| | | | | | |
|---|---|---|---|---|---|
| EWSR1 OT | SEQ ID | AGGTGCCCTGTTCCATGCT | EWSR1 OT rv | SEQ ID | AGGTGCCCTGTTCCATGCT |
| fw | NO: 44 | | | NO: 45 | |
| FLI1 OT fw | SEQ ID | GTGAGTTTACCTTGGCCTGC | Int8 Fli1 rv | SEQ ID | GTGAGTTTACCTTGGCCTGC |
| | NO: 46 | | | NO: 47 | |

qPCR primers

| | | | | | |
|---|---|---|---|---|---|
| qEWSF1 fw | SEQ ID | CCCAGCCAGATCCGTATCAG | qEWSFLI rv | SEQ ID | GCAGCTCCAGGAGGAATTG |
| | NO: 48 | | | NO: 49 | |
| qEWSF2 fw | SEQ ID | GCAGGGCTACAGTGCTTAC | | | |
| | NO: 50 | | | | |
| GAPDH fw | SEQ ID | ACCACAGTCCATGCCATCA | GAPDH rv | SEQ ID | TCCACCACCCTGTTGCTGTA |
| | NO: 52 | | | NO: 53 | |

RT-PCR BA deletion detection primers

| | | | | | |
|---|---|---|---|---|---|
| qBCR-ABL | SEQ ID | GATGCCAAGGATCCAACGAC | qBCR-ABL rv | SEQ ID | GGCTTCACACCATTCCCCAT |
| fw | NO: 54 | | | NO: 55 | |

PCR/RT-PCR controls primers

| | | | | | |
|---|---|---|---|---|---|
| Albumin fw | SEQ ID | GCTGTCATCTCTTGTGGGCTGT | Albumin rv | SEQ ID | ACTCATGGGAGCTGCTGGTTC |
| | NO: 56 | | | NO: 57 | |
| GAPDH fw | SEQ ID | ACCACAGTCCATGCCATCA | GAPDH rv | SEQ ID | TCCACCACCCTGTTGCTGTA |
| | NO: 58 | | | NO: 59 | |

Deep sequencing primers

| | | | | | |
|---|---|---|---|---|---|
| EWSR1 ON | SEQ ID | CTTTCCCTACACGACGCTCTTCCGAT | EWSR1 ON | SEQ ID | GACTGGAGTTCAGACGTGTGCTCTTCC |
| NGS fw | NO:60 | CTAGGTGCCCTGTTCCATGCT | NGS rv | NO: 61 | GATCTTGGCCTAGGCTTTTCAACAGA |
| EWSR1 offT1 | SEQ ID | CTTTCCCTACACGACGCTCTTCCGAT | EWSR1 offT1 | SEQ ID | GACTGGAGTTCAGACGTGTGCTCTTCC |
| NGS fw | NO: 62 | CTATGGTATTCTCACGCTGCCA | NGS rv | NO: 63 | GATCTTGGAGATGAATGGGAAGCGAA |
| EWSR1 offT2 | SEQ ID | CTTTCCCTACACGACGCTCTTCCGAT | EWSR1 offT3 | SEQ ID | GACTGGAGTTCAGACGTGTGCTCTTCC |
| NGS fw | NO: 64 | CTTCCCACTTGTTTATTCCTCTGTG | NGS rv | NO: 65 | GATCTATTCCAGAGAAGGACATTGCCA |
| EWSR1 offT3 | SEQ ID | CTTTCCCTACACGACGCTCTTCCGAT | EWSR1 offT3 | SEQ ID | GACTGGAGTTCAGACGTGTGCTCTTCC |
| NGS fw | NO: 66 | CTTGGGAGTTCTCTAAGGCTGC | NGS rv | NO: 67 | GATCTGTGACTTTTCCCACCGCCTC |
| EWSR1 offT4 | SEQ ID | CTTTCCCTACACGACGCTCTTCCGAT | EWSR1 offT4 | SEQ ID | GACTGGAGTTCAGACGTGTGCTCTTCC |
| NGS fw | NO: 68 | CTCTCCTTTTCTCCTCCTGCCAGC | NGS rv | NO: 69 | GATCTGACTTGGATCTTCAACCGCC |
| EWSR1 offT5 | SEQ ID | CTTTCCCTACACGACGCTCTTCCGAT | EWSR1 offT5 | SEQ ID | GACTGGAGTTCAGACGTGTGCTCTTCC |
| NGS fw | NO: 70 | CTAGCTTGCTATTCTTTGAGATGAAC | NGS rv | NO: 71 | GATCTTGAATAAAGGCCCCGATGACC |
| EWSR1 offT6 | SEQ ID | CTTTCCCTACACGACGCTCTTCCGAT | EWSR1 offT6 | SEQ ID | GACTGGAGTTCAGACGTGTGCTCTTCC |
| NGS fw | NO: 72 | CTCCTGGGTTGTAACTGTGGGT | NGS rv | NO: 73 | GATCTTTCTGGGAGTCGTAGGCTTAGT |
| EWSR1 offT7 | SEQ ID | CTTTCCCTACACGACGCTCTTCCGAT | EWSR1 offT7 | SEQ ID | GACTGGAGTTCAGACGTGTGCTCTTCC |
| NGS fw | NO: 74 | CTCTCGGGCCTGTTCCTTCATA | NGS rv | NO: 75 | GATCTCCACCTCCAGAAGCCCTTAG |
| EWSR1 offT8 | SEQ ID | CTTTCCCTACACGACGCTCTTCCGAT | EWSR1 offT8 | SEQ ID | GACTGGAGTTCAGACGTGTGCTCTTCC |
| NGS fw | NO: 76 | CTGCAAGAATTTCAAGGCCCCAG | NGS rv | NO: 77 | GATCTAGGGATGACTTGACTGCTGA |
| EWSR1 offT9 | SEQ ID | CTTTCCCTACACGACGCTCTTCCGAT | EWSR1 offT9 | SEQ ID | GACTGGAGTTCAGACGTGTGCTCTTCC |
| NGS fw | NO: 78 | CTGTCACTCACCTGGCTGCTTC | NGS rv | NO: 79 | GATCTAGCATTCCTCATTTGATTCCAGA |
| FLI1 ON | SEQ ID | CTTTCCCTACACGACGCTCTTCCGAT | FLI1 ON | SEQ ID | GACTGGAGTTCAGACGTGTGCTCTTCCG |
| NGS fw | NO: 80 | CTGTTGTCTCCCGCATGCCAG | NGS rv | NO: 81 | ATCTGGAATGGGTAGGCAGAGTC |
| FLI1 offT1 | SEQ ID | CTTTCCCTACACGACGCTCTTCCGAT | FLI1 offT1 | SEQ ID | GACTGGAGTTCAGACGTGTGCTCTTCCG |
| NGS fw | NO: 82 | CTAGGGAGGGTCTAATCTAGGAGC | NGS rv | NO: 83 | ATCTCCCTCTTCCCCACCATTTTGT |
| FLI1 offT2 | SEQ ID | CTTTCCCTACACGACGCTCTTCCGAT | FLI1 offT2 | SEQ ID | GACTGGAGTTCAGACGTGTGCTCTTC |
| NGS fw | NO: 84 | CTGCATACAGGGCTTCTTTCGTG | NGS rv | NO: 85 | CGATCTCGTTCTTCCTGTGCCATCCT |
| FLI1 offT3 | SEQ ID | CTTTCCCTACACGACGCTCTTCCGAT | FLI1 offT3 | SEQ ID | GACTGGAGTTCAGACGTGTGCTCTTC |
| NGS fw | NO: 86 | CTTGTGTGGAGGAGGGAGTCAA | NGS rv | NO: 87 | CGATCTGGCCTTCAGAACTCATCAAGG |
| FLI1 offT4 | SEQ ID | CTTTCCCTACACGACGCTCTTCCGAT | FLI1 offT4 | SEQ ID | GACTGGAGTTCAGACGTGTGCTCTTC |
| NGS fw | NO: 88 | CTATCCTCACAGAGCATTGCAG | NGS rv | NO: 89 | CGATCTGTACTGATTCTGGGGCTTGCT |
| FLI1 offT5 | SEQ ID | CTTTCCCTACACGACGCTCTTCCGAT | FLI1 offT5 | SEQ ID | GACTGGAGTTCAGACGTGTGCTCTTC |
| NGS fw: | NO: 90 | CTCGTTGGCTGTGTGTCTGTTTC | NGS rv | NO: 91 | CGATCTAGGAGTGGGGAGTCTTTCGT |
| FLI1 offT6 | SEQ ID | CTTTCCCTACACGACGCTCTTCCGAT | FLI1 offT6 | SEQ ID | GACTGGAGTTCAGACGTGTGCTCTTC |
| NGS fw: | NO: 92 | CTAGGAGACCGATGGACAGACG | NGS rv | NO: 93 | CGATCTCCTCCCTCCTTTCCCCTGAC |
| FLI1 offT7 | SEQ ID | CTTTCCCTACACGACGCTCTTCCGAT | FLI1 offT7 | SEQ ID | GACTGGAGTTCAGACGTGTGCTCTTC |
| NGS fw: | NO: 94 | CTTCCATAAGTTGACTCTGGCAGG | NGS rv | NO: 95 | CGATCTAGAGTGCCTTGGTCAAATGG |
| FLI1 offT8 | SEQ ID | CTTTCCCTACACGACGCTCTTCCGAT | FLI1 offT8 | SEQ ID | GACTGGAGTTCAGACGTGTGCTCTTC |
| NGS fw: | NO: 96 | CTAGTGTTGGGATTACAGGCGTG | NGS rv | NO: 97 | CGATCTGCCTGGGAATTTCACTGTGCC |
| FLI1 offT9 | SEQ ID | CTTTCCCTACACGACGCTCTTCCGAT | FLI1 offT9 | SEQ ID | GACTGGAGTTCAGACGTGTGCTCTTC |
| NGS fw: | NO: 98 | CTAGTTCCCCTCTCCTCCCTG | NGS rv | NO: 99 | CGATCTCACTTCCCATGGACAGCTTG |
| FLI1 offT10 | SEQ ID | CTTTCCCTACACGACGCTCTTCCGAT | FLI1 offT10 | SEQ ID | GACTGGAGTTCAGACGTGTGCTCTTC |
| NGS fw: | NO: 100 | CTGAGGGGTAAAGGATTGGAGCC | NGS rv | NO: 101 | CGATCTCGGAGAGATTGAAGGGAGCG |

crRNAs

| | | |
|---|---|---|
| EWSR1- | SEQ ID | GTCATAAGAAGGGTTCTGCTGCCCGT |
| FLI1 type I | NO: 102 | AG |
| EWSR1- | SEQ ID | GGCCAGCAGTGAACTCTGCTGCCCGT |
| FLI1 type | NO: 103 | AG |
| II | | |
| BCR-ABL | SEQ ID | CCGCTGAAGGGCTTTTGAACTCTGCT |
| | NO: 104 | TA |

A couple of sgRNAs were designed for each intronic region using the crispr.mit.edu/ and benchling.com/crispr webtools following the standard sgRNA design principles: making sure to pick targeting sequences that are upstream of a PAM sequence, unique to the target compared to the rest of the genome, and selecting those with as few predicted off-target events as possible. The sgRNAs, sgEWSR13.2 (hereafter sgE3.2 (SEQ ID NO: 2)) and sgFLI18.2 (sgF8.2 (SEQ ID NO: 3)) were cloned in the pLVX-U6E3.2-H1F8.2-Cas9-2A-eGFP (hereafter pLV-U6EH1F-C9G) that drives similar sgRNA expression levels from two different RNA polymerase III promoters (U6 and H1) and a simultaneously regulated expression of Cas9 and GFP proteins by a 2A self-cleaving peptide (FIG. 1d). Cleavage of introns 3 and 8 of EWSR1 and FLI1, respectively, should result in a deletion of 27.67 kb, removing a critical portion of the EWSR1 transactivation domain, and together with a frameshift alteration of the entire FLI1 DNA binding domain (FIGS. 1b and 2). SEQ ID NO: 6 is the amino acid sequence of the EWSR1-FLI1 chimeric protein and SEQ ID NO: 7 is the amino acid sequence of the edited EWSR1-FLI1 truncated protein, both represented in FIG. 2. A673 cells were transduced with the pLV-U6EH1F-C9G vector and total genomic DNA was isolated at 2, 4 and 6 days post-transduction (pt) for subsequent analysis. 2 days pt DNA deep sequencing analysis showed indel frequencies of 61.8 in EWSR1 and 66.2% in FLI1 on-target sites (table 3).

Table 3. NGS analysis of the on-target EWSR1 and FLI1 sites. a,c. Summary of the EWSR1 and FLI1 loci analysis (sgRNA sequence, chromosome position, total reads and efficiency). b,d. Indels at EWSR1 and FLI1 loci in induced A673 edited cells. Wild-type (WT) sequences are listed at the top of each figure. sgRNA sequence is underlined Identified mutations are shown in bold font. −, deletion.

a. (SEQ ID NO: 2)

| | | | | Control | | | Edited | | |
|---|---|---|---|---|---|---|---|---|---|
| | sgRNA | | | | | Editing | | | Editing |
| | sequence | | | Total | Modified | efficiency | Total | Modified | efficiency |
| On target | (5′-3′) | Chromosome | Position | reads | Reads | (%) | reads | Reads | (%) |
| sgEWS3.2 | TGGTTGCACA | 22 | 29272832 | 49650 | 0 | 0.0 | 22981 | 14207 | 61.8 |
| | GTAAGTGGCG | | | | | | | | |

b. (SEQ ID NOs: 8 to 19)

| Sequence | Reads |
|---|---|
| GCAGTGCATAGATATTAAGTAACTTGCCAGTGGTTGCACAGTAAGTGGCGGGGTTAGCTCTAAAAACTGGCGACCTAGCCAA | x7858 |
| GCAGTGGATAGATATTAACTAACTTCCCACTCGTTCCACACTAAGT-CCGGGGTTAGGTCTAAAAACTGCCCACCTAGCCAA | x1459 |
| GCAGTGCATAGATATTAAGTAACTTGCCAGTGGTTGCACAGTAAGTGTGGCGGGGTTAGTTCTAAAAACTGGCGACCTAGCCAA | x835 |
| GCAGTGCATAGATATTAAGTAACTTGCCAGTGGTTGCACAGTAAGTGGGCGGGGTTAGCTCTAAAAACTGGCGACCTAGCCAA | x763 |
| GCAGTGCATAGATATTAAGTAACTTGCCAGTGGTTGCACAGTAA---GCGGGGTTAGCTCTAAAAACTAGCGACCTAGCCAA | x688 |
| GCAGTGCATAGATATTAAGTAACTTGCCAGTGGTT------------GCGGGGTTAGCTCTAAAAACTAGCGACCTAGCCAA | x526 |
| GCAGTGCATAGATATTAAGTAACTTGCCAGTGGTTGCACAGTAA---GGCGGGGTTAGCTCTAAAAACTGGCGACCTAGCCAA | x446 |
| GCAGTCCATACATATTAAGTAACTTCCC---------------ACTGGGCCGGTTACCTCTAAAAACTGGCGACCTAGCCAA | x343 |
| GCAGTGCATAGATATTAAGTAACTTGCCAGTGGTTGCACA-------GCGGGGTTAGCTCTAAAAACTGGCGACCTAGCCAA | x286 |
| GCACTCCATAGATATTAACTAACTTCCCACTGGTTCCACA------------CTTAGCTCTAAAAACTGCCCACCTAGCCAA | x254 |
| GCAGTGCATAGATATTAAGTAACTTGCCAGT--------------------GGTTAGCTCTAAAAACTGGCGACCTAGCCAA | x218 |
| GCAGTGCATAGATATTAAGTAACTTGCCAGTGGTTGCACAGTA----GCGGGGTTAGCTCTAAAAACTGGCGACCTAGCCAA | x213 |

c. (SEQ ID NO: 3)

| | | | | Control | | | Edited | | |
|---|---|---|---|---|---|---|---|---|---|
| | sgRNA | | | | | Editing | | | Editing |
| | sequence | | | Total | Modified | efficiency | Total | Modified | efficiency |
| On target | (5′-3′) | Chromosome | Position | reads | Reads | (%) | reads | Reads | (%) |
| sgFLI8.2 | AGTGGGCCAC | 11 | 128809772 | 1461 | 0 | 0.0 | 12315 | 8151 | 66.2 |
| | ACTGCGACAA | | | | | | | | |

d. (SEQ ID NOs: 20 to 31)

| Sequence | Reads |
|---|---|
| TGGGTAGGCAGAGTCCCTGGGATGGGAAGGTGAGTGGGCCACACTGCGACAAGGGCCTGCTAGCTCCCAATCTCGATGGACTT | x3792 |
| TGGGTAGGCAGAGTCCCTGGGATGGGAAGGTGAGTGGGCCACACTGCGAACAAGGGCCTGCTAGCTCCCAATCTCGATGGACTT | x1111 |
| TGGGTAGGCAGAGTCCCTGGGATGGGAAGGTGAGTGGGCCACACTGC-------------TAGCTCCCAATCTCGATGGACTT | x622 |
| TGGGTAGGCAGAGTCCCTGGGATGGGAAGGTGAGTGGGCC-----------------TGCTAGCTCCCAATCTCGATGGACTT | x515 |
| TGGGTAGGCAGAGTCCCTGGGATGGGAAGGTGAGTGGGCCACACTGCGA-AAGGGCCTGCTAGCTCCCAATCTCGATGGACTT | x508 |
| TGGGTAGGCAGAGTCCCTGGGATGGGAAGGTGAGTGGGCCACACTGCGA---GGGCCTGCTAGCTCCCAATCTCGATGGACTT | x411 |
| TGGGTAGGCAGAGTCCCTGGGATGGGAAGGTGAGTGGGCCACACTGC---AAGGGCCTGCTAGCTCCCAATCTCGATGGACTT | x326 |
| TGGGTAGGCAGAGTCCCTGGGATGGGAAGGTGAGTGGGCCACACTGCGA--AGGGGCTGCTAGCTCCCAATCTCCATGGACTT | x187 |
| TGGGTAGGCAGAGTCCCTGGGATGGGAAGGTGAGTGGGCCACACTGCGA---------GCTAGCTCCCAATCTCCATGGACTT | x191 |
| TGGGTAGGCAGAGTCCCTGGGATGGGAAGGTGAGTGGGCCACACTG-----------------CTCCCAATCTCCATGGACTT | x173 |
| TGGGTAGGCAGAGTCCCTGGGATGGGAAGGTGAGTGGGCCACACTGCG-CAAGGGCCTGCTAGCTCCCAATCTCGATGGACTT | x155 |
| TGGGTAGGCAGAGTCCCTGGGATGGGAAGGTGAGTGGGCCACA--------AGGGCCTGCTAGCTCCCAATCTCGATGGACTT | x147 |

[symbol]

Targeting the EWSR1-FLI1 Fusion Gene In Vitro

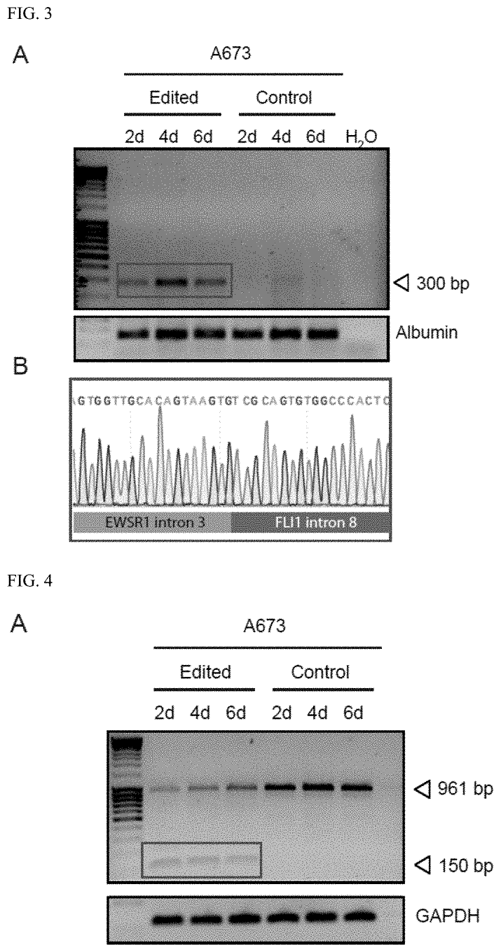

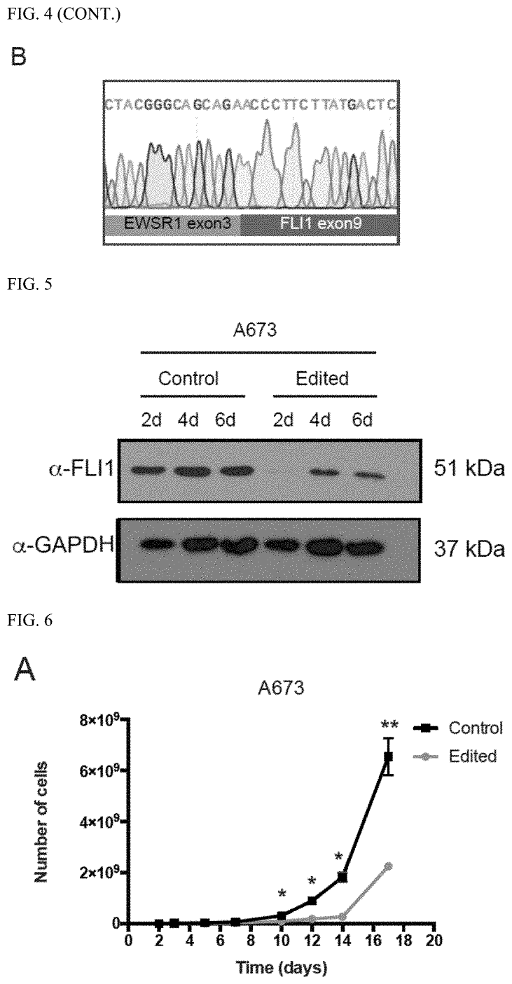

Two ES cell lines, A673 and RD-ES, harbouring respectively the type1 or type2EWSR1-FLI1 isoforms, were chosen as model systems. We first examined the ability of pLV-U6EH1F-C9G to generate EWSR1-FLI1 fusion gene deletions in the A673. Osteosarcoma U2OS cell line, which do not contain the fusion gene and an empty pLV-U6#H1#-C9G vector that do not contain sgRNA sequences were used as cell line and vector controls, respectively. PCR analysis of genomic DNA region spanning the intronic target sites extracted at days 2, 4 and 6 pt revealed a unique 427.67 kb deletion product whose sequence was verified by Sanger sequencing (FIG. 3). Similarly, the simultaneous sgE3.2 and sgF8.2 expression resulted in a robust reduction of EWSR1-FLI1 mRNA and protein, as showed by qRT-PCR and Western blot analysis (FIGS. 4 and 5).

Targeting EWSR1-FLI1 Fusion Gene with a KO Deletion CRISPR-Based Approach Inhibits Cancer Cell Survival, Proliferation and Tumorigenicity In Vitro

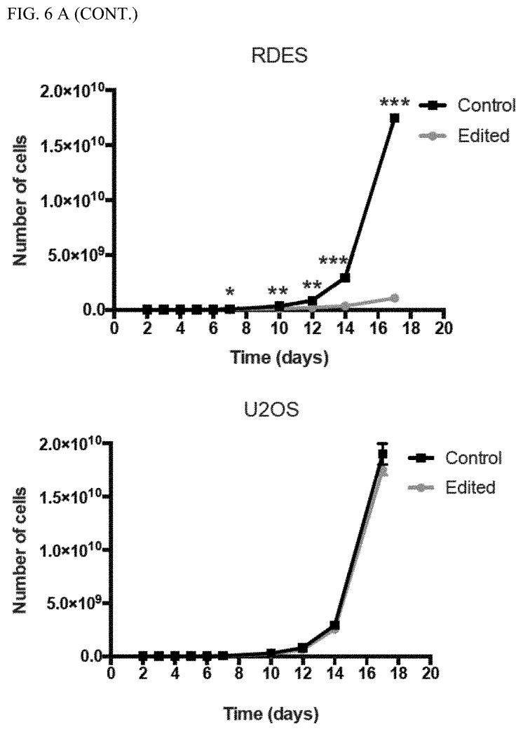

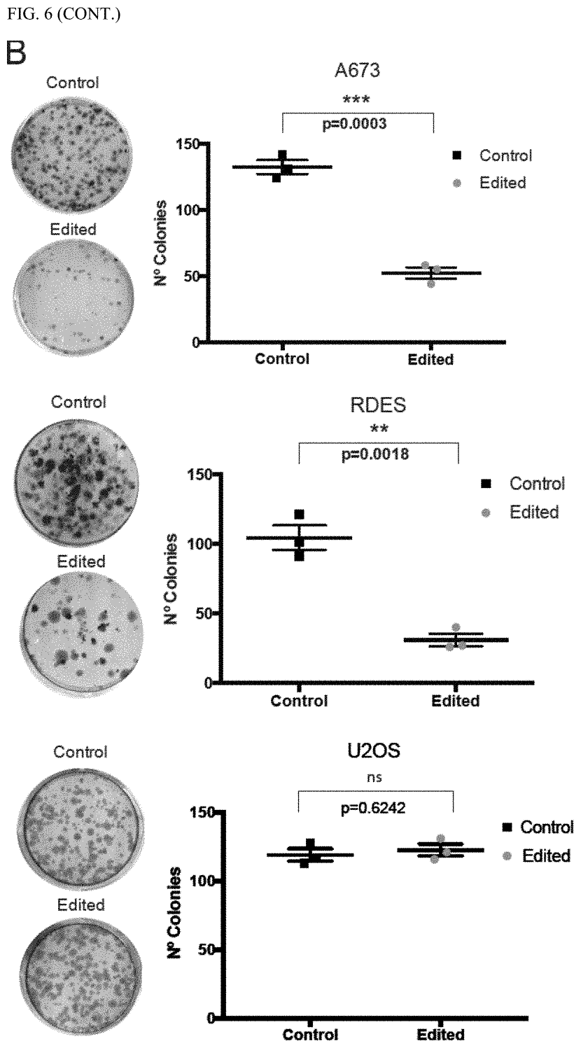

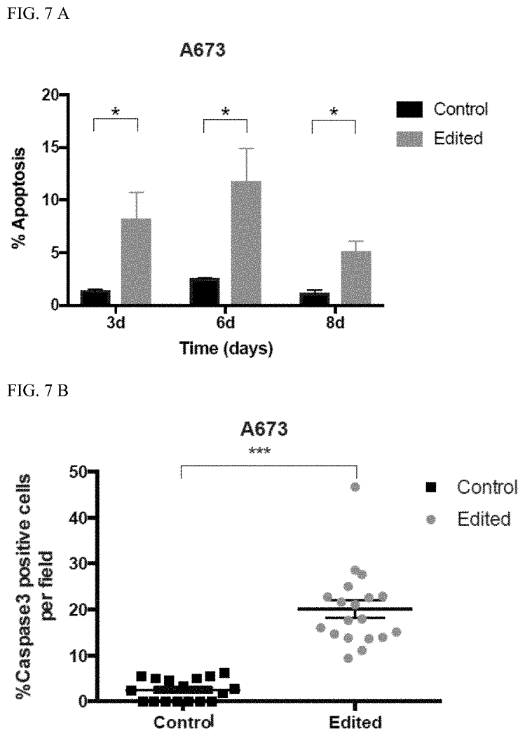

To investigate whether targeted deletion of EWSR1-FLI1 could induce death of cancer cells, cell survival, proliferation and tumorigenicity in vitro assays were conducted. A673, RD-ES and U2OS transduced with pLV-U6EH1F-C9G and control plasmid were subjected to growth rate and colony forming on soft agar assays. The growth rate assay demonstrated that EWSR1-FLI1 deletion in both A673 and RD-ES significantly suppressed cell proliferation compared with their corresponding control cells and has no effect on U2OS cells (FIG. 6a). Furthermore, co-expression of Cas9 and sgRNAs in A673 and RD-ES cells, led to 61.5% and 73.3% significant reductions in the number of colonies in colony forming and soft agar assays, respectively, suggesting that deletion of EWSR1-FLI1 using CRISPR inhibits the survival and tumorigenicity of the ES cancer cells. The expression of Cas9 and sgRNAs in U2OS cells did not alter significantly the number of colonies (FIG. 6b). Taken together, our data show that EWSR1-FLI1 deletion insufficient for tumour suppressor activity, inhibiting cell growth. Notably, we observed that 3, 6 and 8 days pt EWSR1-FLI1 deletion was followed by significant increased cell death, with a peak of 20% of increased cell death at day 6 measured by the number of cells presenting fragmented DNA (subG1 peak) compared with A673-pLV-U6#H1#-C9G control cells (FIG. 7a). Casp3 activation analysis confirmed these results (FIG. 7b). Thus, fusion gene deletion leads to apoptotic death in ES cancer cells.

EWSR1-FLI1 Fusion-Gene Targeting is Highly Specific

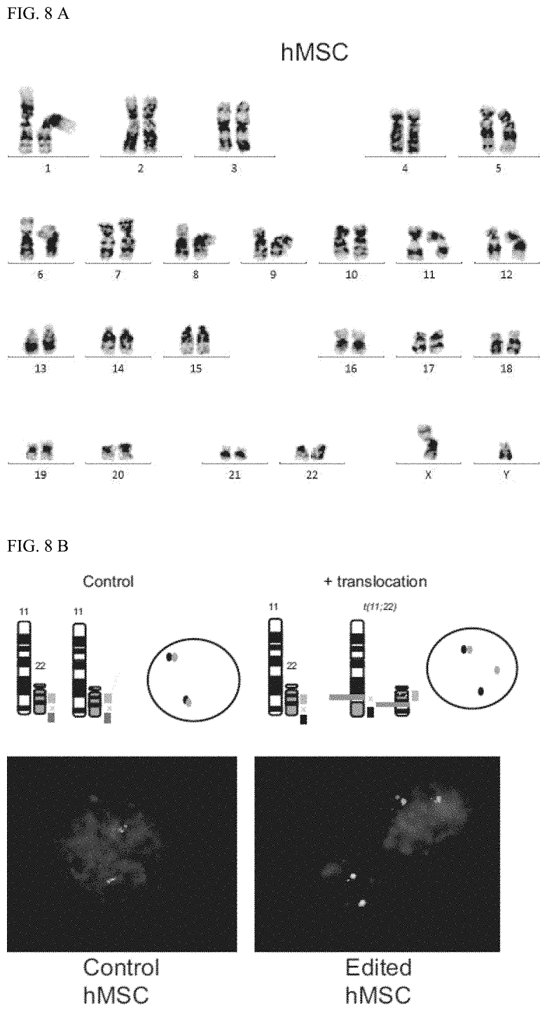

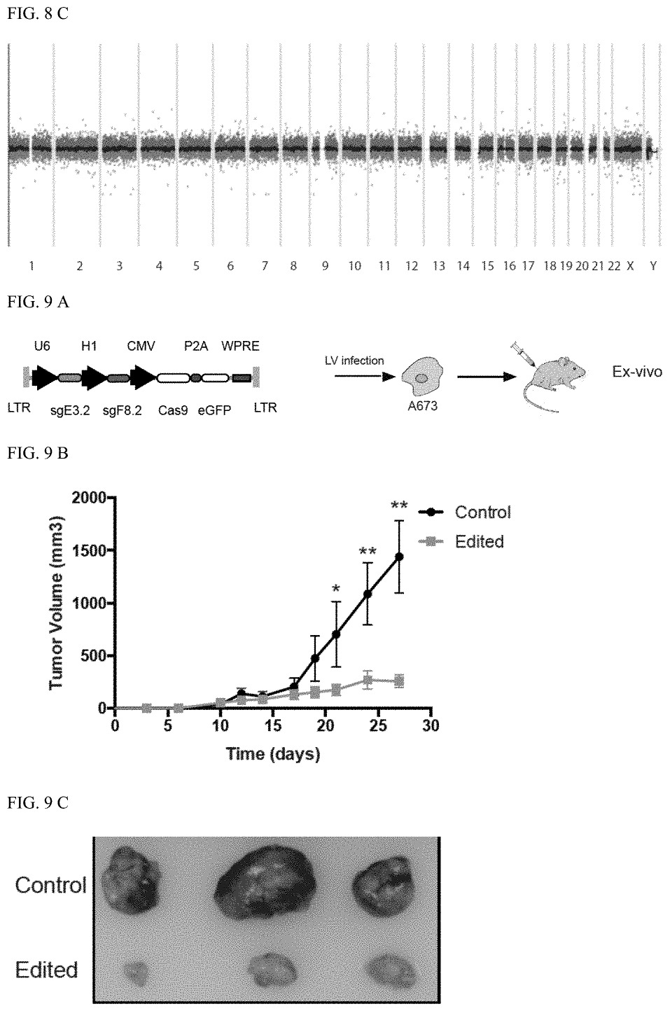

Karyotype and FISH analysis were performed in human mesenchymal stem cells (hMSC) transduced with pLV-U6EH1F-C9G to evaluate whether the cleavage of EWSR1 and FLI1 wild type genes could induce genomic alterations in WT cells. G-banded methaphases showed a normal karyotype (FIG. 8a); and additionally FISH analysis with an EWSR1 break-apart probe showed no altered FISH signals (FIG. 8b). Similarly, high-density array comparative genomic hybridization (aCGH) analysis covering the whole genome showed no copy number variations (CNVs) (FIG. 8c). These data suggest that the therapeutic targeting of the cancer EWSR1-FLI1 fusion gene is highly specific and would not interfere with WT cells. On the other hand, to potentially identify mutations induced at off-target sites we performed next generation sequencing (NGS) of amplicons of 19 genomic regions with the highest homology to the target site (off-target sites 1-9/10 for EWSR1 and FLI1, respectively) at day 7 post-transduction of A673, RD-ES and hMSC cells. None of all read has mutations at the predicted off-target sites (table 4).

Targeted EWSR1-FLI1 Fusion Gene Deletion Induce Partial Remission of Xenografted Tumours

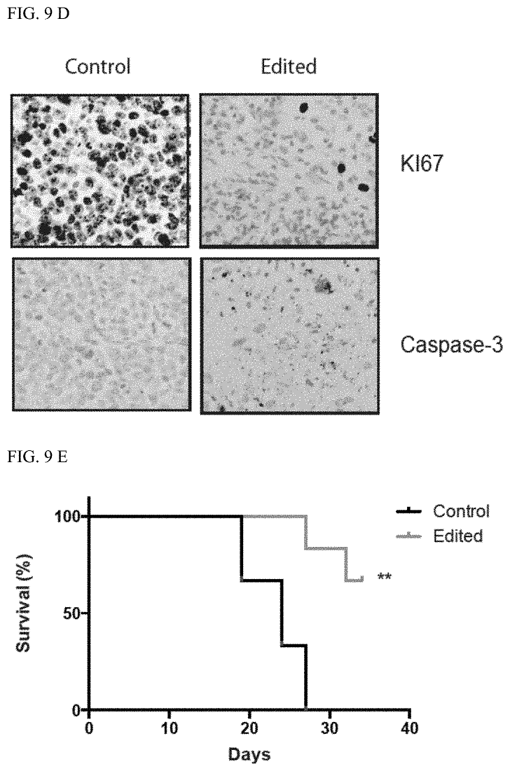

In order to determine the effects of EWSR1-FLI1 deletion in vivo, the flanks of nude mice were subcutaneously implanted with control-pLV-U6#H1#-C9G and pLV-U6EH1F-C9G transduced A673 cells (FIG. 9a). Mice injected with control cells showed an exponential growth of tumours during the 28 days of analysis. In contrast, mice injected with pLV-U6EH1F-C9G cells gave rise to dramatically smaller subcutaneous tumours than those produced by A673 control cells (FIGS. 9b and c). Moreover, ex vivo CRISPR edited cells exhibited a markedly reduced number of viable tumour cells and more extensive necrotic regions in CRISPR edited cells than in controls as shown by H&E (hematoxylin and eosin) staining revealed, a significant 65% reduction of the KI67 proliferation marker and a significant 25% increased level of caspase-3 protein expression compared with control tumours (FIG. 9d). Importantly, mice xenografted with pLV-U6EH1F-C9G cells showed no associated mortality in the 35 days of the study, whereas all controls needed to be sacrificed after two weeks of cell injection (FIG. 9e). These results confirmed the therapeutic efficacy of the EWSR1-FLI1 ex vivo fusion gene edition.

TABLE 4
Indel analysis of the most probable off-target sites with the highest homology to
the on-target sites by amplicon based next-generation sequencing (NGS) analysis at day
7 pt of hMSC, A673 and RDES cells. On-target sequences are listed at the top of each
pannel. Differences in nucleotides with on-target sequence are shown in bold font. None
of all read has mutations at the predicted off-target sites.

| | | | | | 2H WT | | | 2H EF | | |
|---|---|---|---|---|---|---|---|---|---|---|
| | | | | | | | Editing | | | Editing |
| OFF | SEQ | | | | | Modi | ef- | | Modi- | ef- |
| Target | ID | sgRNA sequence | Chromo- | | Total | fied | ficiency | Total | fied | ficiency |
| 2H: | NO: | (5′-3′) | some | Position | reads | Reads | (%) | reads | Reads | (%) |
| sgEWS3.2 | 105 | TGGTTGCACAGTAAGTGGCG | | | | | | | | |
| OFT#1 | 106 | TGATTGCACTGTAAGTGGCC | chr5 | −78568232 | 49650 | 2 | 0.0 | 23200 | 5 | 0.0 |
| OFT#2 | 107 | TAGGTGCTCAGTAAGTGGCT | chr10 | −35792862 | 21646 | 0 | 0.0 | 21646 | 1 | 0.0 |
| OFT#3 | 108 | CGACTTCACAGTAAGTGGCG | chr18 | −74446599 | 39768 | 0 | 0.0 | 39768 | 0 | 0.0 |
| OFT#4 | 109 | AGGTAGCGCAGTTAGTGGCG | chr9 | 14347518 | 47792 | 0 | 0.0 | 31603 | 0 | 0.0 |
| OFT#5 | 110 | AGGTAGAACAGTAAGTGGCA | chr1 | 194593114 | 3010 | 0 | 0.0 | 27290 | 3 | 0.0 |
| OFT#6 | 111 | TAGTTGTTCAGTAAGTGGCA | chr1 | −11805340 | 47295 | 5 | 0.0 | 47691 | 3 | 0.0 |
| OFT#7 | 112 | GGGTAGCAGAGGAAGTGGCG | chr19 | 32779534 | 2985 | 0 | 0.0 | 40221 | 3 | 0.0 |
| OFT#8 | 113 | TGGATGCTGAGTAAGTGGCC | chrX | 115433334 | 550 | 0 | 0.0 | 37235 | 3 | 0.0 |
| OFT#9 | 114 | TGCTTGCAAGGTAAGTGGCC | chr2 | 162011934 | 2487 | 0 | 0.0 | 36328 | 6 | 0.0 |
| sgFLI8.2 | 115 | AGTGGGCCACACTGCGACAA | | | | | | | | |
| OFT#1 | 116 | GATTGGCCACACTGTGACAA | chr9 | −124911364 | 30160 | 8 | 0.0 | 29771 | 10 | 0.0 |
| OFT#2 | 117 | GGCGGGGCACACAGCGACAA | chr1 | 20716449 | 57401 | 2 | 0.0 | 23356 | 0 | 0.0 |
| OFT#3 | 118 | AGTGAGGAACACTGCGGCAA | chr6 | 30452057 | 988 | 0 | 0.0 | 25067 | 0 | 0.0 |
| OFT#4 | 119 | TGTGGGCCAGGCTGCGGCAA | chr14 | 58819512 | 480 | 0 | 0.0 | 31988 | 2 | 0.0 |
| OFT#5 | 120 | AGTGGGCTAGCCTGCGACAG | chr10 | 3167786 | 1157 | 0 | 0.0 | 28473 | 2 | 0.0 |
| OFT#6 | 121 | AGTGGGGCTGTCTGCGACAA | chr11 | 793219 | 27414 | 0 | 0.0 | 13965 | 2 | 0.0 |
| OFT#7 | 122 | TGTGAACCACACTGTGACAA | chrX | 126483786 | 30402 | 0 | 0.0 | 7328 | 0 | 0.0 |
| OFT#8 | 123 | AGTGTGCTCCACTGTGACAA | chr18 | 23078807 | 632 | 0 | 0.0 | 724 | 0 | 0.0 |
| OFT#9 | 124 | CGTGGGCCAGCCTGGGACAA | chrX | −153625648 | 19552 | 2 | 0.0 | 8569 | 0 | 0.0 |
| OFT#10 | 125 | AGAGAGCCACACTGAGACAG | chr5 | 133765919 | 64679 | 0 | 0.0 | 34737 | 13 | 0.0 |
| sgEWS3.2 | 105 | TGGTTGCACAGTAAGTGGCG | | | | | | | | |
| OFT#1 | 106 | TGATTGCACTGTAAGTGGCC | chr5 | −78568232 | 1542 | 0 | 0.0 | 16010 | 1 | 0.0 |
| OFT#2 | 107 | TAGGTGCTCAGTAAGTGGCT | chr10 | −35792862 | 21610 | 0 | 0.0 | 32765 | 0 | 0.0 |
| OFT#3 | 108 | CGACTTCACAGTAAGTGGCG | chr18 | −74446599 | 26865 | 0 | 0.0 | 6309 | 0 | 0.0 |
| OFT#4 | 109 | AGGTAGCGCAGTTAGTGGCG | chr9 | 14347518 | 12091 | 0 | 0.0 | 19888 | 0 | 0.0 |
| OFT#5 | 110 | AGGTAGAACAGTAAGTGGCA | chr1 | 194593114 | 4982 | 0 | 0.0 | 28486 | 4 | 0.0 |
| OFT#6 | 111 | TAGTTGTTCAGTAAGTGGCA | chr1 | −11805340 | 31235 | 2 | 0.0 | 60048 | 2 | 0.0 |
| OFT#7 | 112 | GGGTAGCAGAGGAAGTGGCG | chr19 | 32779534 | 42436 | 4 | 0.0 | 62331 | 2 | 0.0 |
| OFT#8 | 113 | TGGATGCTGAGTAAGTGGCC | chrX | 115433334 | 36814 | 0 | 0.0 | 60048 | 2 | 0.0 |
| OFT#9 | 114 | TGCTTGCAAGGTAAGTGGCC | chr2 | 162011934 | 34218 | 9 | 0.0 | 30383 | 0 | 0.0 |
| sgFLI8.2 | 115 | AGTGGGCCACACTGCGACAA | | | | | | | | |
| OFT#1 | 116 | GATTGGCCACACTGTGACAA | chr9 | −124911364 | 35337 | 4 | 0.0 | 46550 | 7 | 0.0 |
| OFT#2 | 117 | GGCGGGGCACACAGCGACAA | chr1 | 20716449 | 7774 | 0 | 0.0 | 1324 | 0 | 0.0 |
| OFT#3 | 118 | AGTGAGGAACACTGCGGCAA | chr6 | 30452057 | 40653 | 4 | 0.0 | 9737 | 2 | 0.0 |
| OFT#4 | 119 | TGTGGGCCAGGCTGCGGCAA | chr14 | 58819512 | 32163 | 0 | 0.0 | 40165 | 0 | 0.0 |
| OFT#5 | 120 | AGTGGGCTAGCCTGCGACAG | chr10 | 3167786 | 39513 | 0 | 0.0 | 8248 | 9 | 0,1 |
| OFT#6 | 121 | AGTGGGGCTGTCTGCGACAA | chr11 | 793219 | 21076 | 2 | 0.0 | 2091 | 0 | 0.0 |
| OFT#7 | 122 | TGTGAACCACACTGTGACAA | chrX | 126483786 | 14647 | 0 | 0.0 | 22616 | 0 | 0.0 |
| OFT#8 | 123 | AGTGTGCTCCACTGTGACAA | chr18 | 23078807 | 2820 | 0 | 0.0 | 3019 | 0 | 0.0 |
| OFT#9 | 124 | CGTGGGCCAGCCTGGGACAA | chrX | −153625648 | 10867 | 0 | 0.0 | 6433 | 0 | 0.0 |
| OFT#10 | 125 | AGAGAGCCACACTGAGACAG | chr5 | 133765919 | 26725 | 9 | 0.0 | 5907 | 0 | 0.0 |
| sgEWS3.2 | 105 | TGGTTGCACAGTAAGTGGCG | | | | | | | | |
| OFT#1 | 106 | TGATTGCACTGTAAGTGGCC | chr5 | −78568232 | 14507 | 0 | 0.0 | 22462 | 3 | 0.0 |
| OFT#2 | 107 | TAGGTGCTCAGTAAGTGGCT | chr10 | −35792862 | 32140 | 0 | 0.0 | 22019 | 5 | 0.0 |
| OFT#3 | 108 | CGACTTCACAGTAAGTGGCG | chr18 | −74446599 | 35951 | 4 | 0.0 | 36436 | 0 | 0.0 |
| OFT#4 | 109 | AGGTAGCGCAGTTAGTGGCG | chr9 | 14347518 | 13125 | 0 | 0.0 | 16382 | 5 | 0.0 |
| OFT#5 | 110 | AGGTAGAACAGTAAGTGGCA | chr1 | 194593114 | 5559 | 0 | 0.0 | 27713 | 2 | 0.0 |
| OFT#6 | 111 | TAGTTGTTCAGTAAGTGGCA | chr1 | −11805340 | 57216 | 0 | 0.0 | 53673 | 3 | 0.0 |
| OFT#7 | 112 | GGGTAGCAGAGGAAGTGGCG | chr19 | 32779534 | 27412 | 0 | 0.0 | 40461 | 0 | 0.0 |
| OFT#8 | 113 | TGGATGCTGAGTAAGTGGCC | chrX | 115433334 | 1017 | 0 | 0.0 | 59512 | 2 | 0.0 |
| OFT#9 | 114 | TGCTTGCAAGGTAAGTGGCC | chr2 | 162011934 | 32978 | 0 | 0.0 | 40946 | 0 | 0.0 |
| sgFLI8.2 | 115 | AGTGGGCCACACTGCGACAA | | | | | | | | |
| OFT#1 | 116 | GATTGGCCACACTGTGACAA | chr9 | −124911364 | 23984 | 6 | 0.0 | 24215 | 0 | 0.0 |
| OFT#2 | 117 | GGCGGGGCACACAGCGACAA | chr1 | 20716449 | 2140 | 0 | 0.0 | 42322 | 4 | 0.0 |
| OFT#3 | 118 | AGTGAGGAACACTGCGGCAA | chr6 | 30452057 | 86311 | 0 | 0.0 | 55558 | 5 | 0.0 |
| OFT#4 | 119 | TGTGGGCCAGGCTGCGGCAA | chr14 | 58819512 | 185 | 0 | 0.0 | 31686 | 10 | 0.0 |
| OFT#5 | 120 | AGTGGGCTAGCCTGCGACAG | chr10 | 3167786 | 22928 | 0 | 0.0 | 27899 | 0 | 0.0 |
| OFT#6 | 121 | AGTGGGGCTGTCTGCGACAA | chr11 | 793219 | 9604 | 0 | 0.0 | 30315 | 2 | 0.0 |
| OFT#7 | 122 | TGTGAACCACACTGTGACAA | chrX | 126483786 | 22152 | 10 | 0.0 | 14962 | 0 | 0.0 |
| OFT#8 | 123 | AGTGTGCTCCACTGTGACAA | chr18 | 23078807 | 13586 | 1 | 0.0 | 56914 | 0 | 0.0 |
| OFT#9 | 124 | CGTGGGCCAGCCTGGGACAA | chrX | −153625648 | 14991 | 0 | 0.0 | 20784 | 0 | 0.0 |
| OFT#10 | 125 | AGAGAGCCACACTGAGACAG | chr5 | 133765919 | 40508 | 8 | 0.0 | 30937 | 0 | 0.0 |

Targeting EWSR1-FLI1 Fusion Gene with CRISPR-Cas9 Blocks Tumour Growth in Vivo

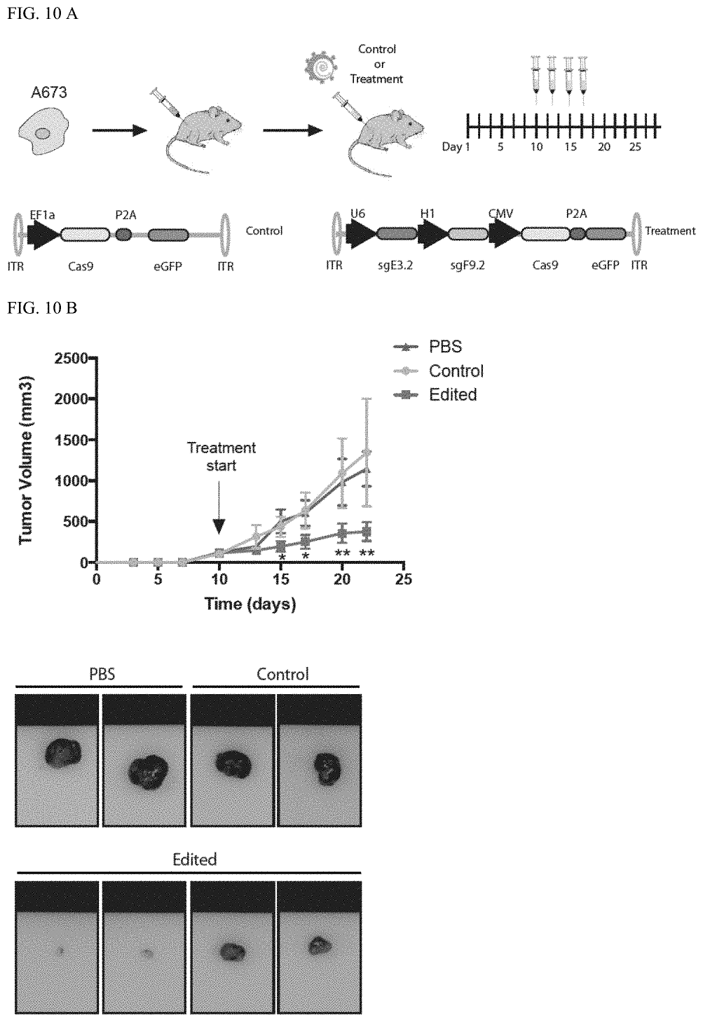

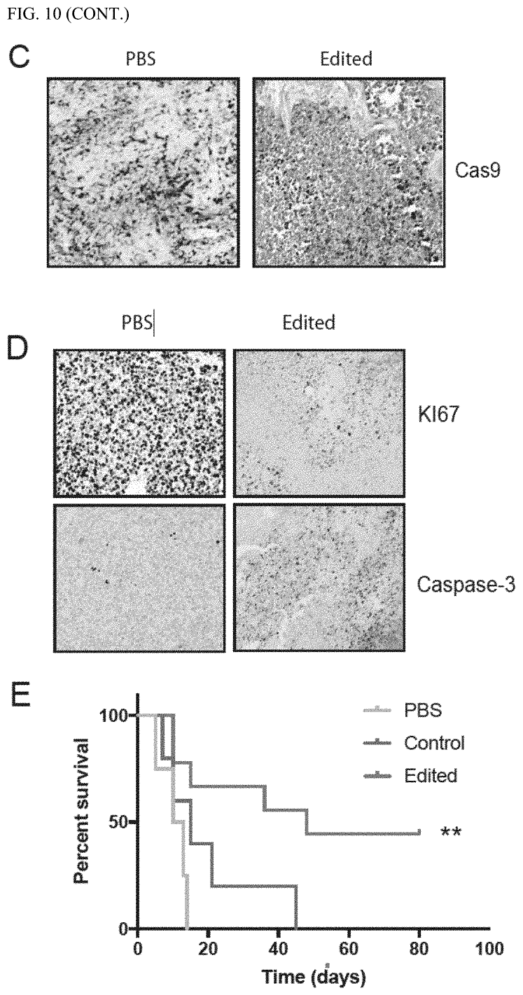

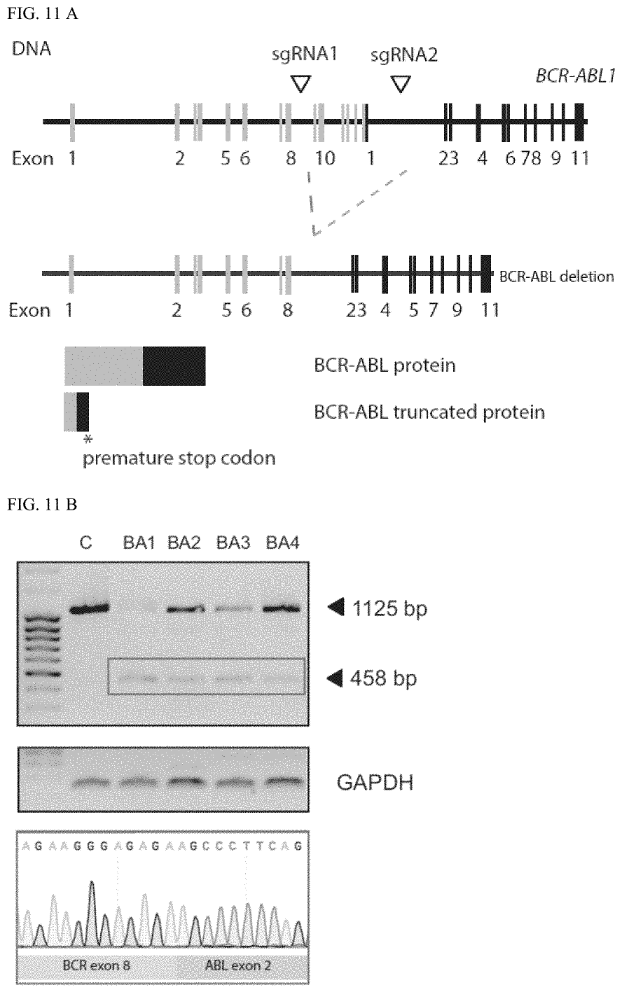

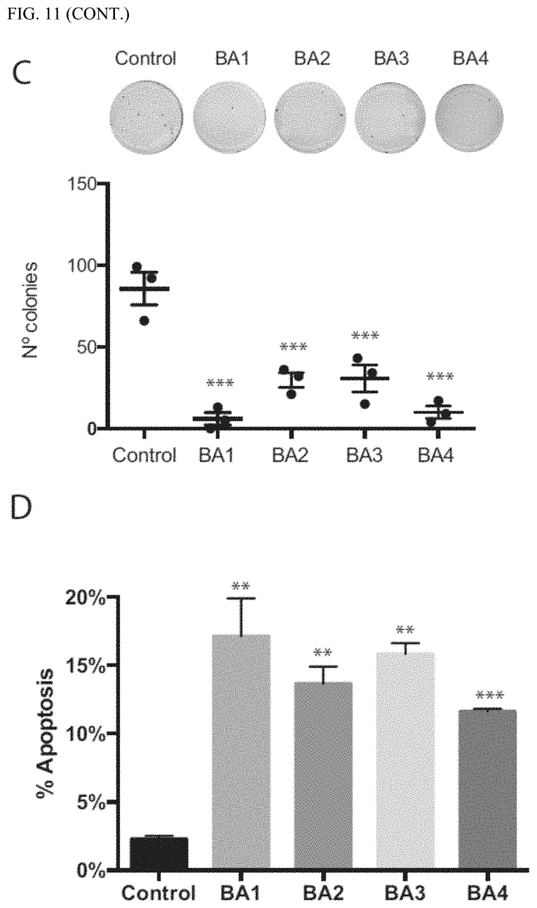

To evaluate whether CRISPR fusion gene deletion can in vivo control human cancer growth in athymic mice we used an adenoviral delivery approach. Wild type A673 cells were subcutaneously injected in the flank of athymic mice (Day 0). The xenografted tumours were allowed to grow for two weeks (Day 10) until reached ˜150 mm³ in size. These tumours were then injected with 2.5×10⁹ plaque-forming units (pfu) of Ad/sgE3.2sgF8.2Cas9, Ad/Cas9 or PBS four times at days 10, 13, 16 and 19 (FIG. 10a). Adenoviral delivery of sgRNAs and the Cas9 nuclease led to significant tumour growth inhibition, resulting in an average tumour size of 298.66 (+92.77) mm³ (P<0.05). Tumour volumes in control groups treated with either PBS or Ad/Cas9 increased over time and reached an average size of 1143.98 (+337.59) mm³ or 1345.25 (+685.16) mm³, respectively, at the end of the treatment (FIG. 10b). Thus, the EWSR1-FLI1 edited experimental cells reduced tumour size by 83.5% compared to the controls, respectively. Immunohistochemic staining using Cas9 antibody confirmed expression of Cas9 protein in tumours injected with Ad/sgEWSR1-sgFLI1-Cas9 (FIG. 10c). The antitumor efficacy of the fusion gene deletion approach was further investigated by histological and immunohistochemical analysis. H&E staining revealed a markedly reduced number of viable tumour cells and more extensive necrotic regions in CRISPR edited cells than in controls. Moreover, CRISPR edited cells exhibited 80% lower levels of KI67 proliferation marker compared with control tumours (FIG. 10d). Fusion gene deleted tumours showed a ≈30% increase in caspase-3 protein expression compared with control tumours (FIG. 11d). Importantly, mice treated with Ad/sgE3.2sgF8.2Cas9 showed no associated mortality in the 80 days of the study, whereas all controls needed to be sacrificed after two weeks of cell injection (FIG. 10e). These results confirmed the therapeutic efficacy of the fusion gene edition approach to treat in vivo tumours driven by EWSR1-FLI1 fusion gene.

Strategy for Targeted of the Chronic Myeloid Leukaemia Tyrosine Kinase BCR-ABL Fusion Gene in K562 Cells

To evaluate whether such gene editing approach might be used as a universal approach for fusion gene driver cancer treatment, we reproduced a similar strategy to delete a classical tyrosine kinase fusion gene. The inventors choose BCR-ABL1 generated by the t(9;22)(q34;q11) translocation, genetic abnormality hallmark of CIVIL. BCR-ABL1 creates a constitutively active tyrosine kinase, which leads to uncontrolled proliferation. We followed the same methodological approach described above. Briefly, four pairs of sgRNAs targeting BCR intron 8 and ABL intron 1 regions were designed (FIG. 11a and Table 2). Following NHEJ these would result in deletion of 133.9 kb of genomic BCR-ABL1 DNA. Successful deletion will remove a large portion of the BCR Db1 homology (DH) domain, together with the frameshift alteration of the entire ABL1 DNA binding domain. Four sgRNAs combinations were cloned to generate the pLV-U6BH1A-C9G (BA1, BA2, BA3 and BA4) vectors. Then, we examined the efficiency to generate BCR-ABL1 targeted deletion in CML patient derived K562 cells that harbour the p210 isoform of the BCR-ABL1 fusion gene. The efficient reduction of BCR-ABL mRNA was confirmed 24 h post-nucleofection by RT-PCR and Sanger sequencing (FIG. 11b). Colony forming assays on metilcellulose agar confirmed that targeted deletion of BCR-ABL1 induced dramatic reduction on proliferation and death in K562 cells. Quantitative data analysis showed that the BCR-ABL1 deletion significantly suppressed colony formation to approximately 85% (FIG. 11c). Quantitative data analysis of SubG1 analysis with the four combinations of sgRNAs in K562 experimental and control cells after 72 h in culture showed significant increase of apoptosis in the BCR-ABL1 deleted cells compared to control cells (P<0.05). (FIG. 11d).

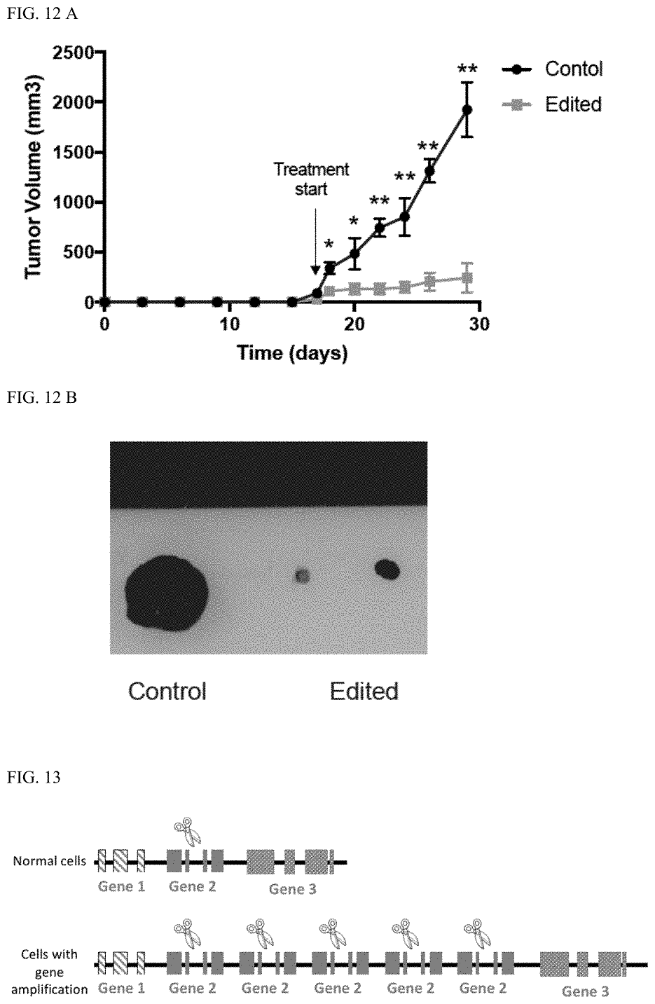

To evaluate BCR-ABL1 deletion effects in vivo, K562 cells were subcutaneously injected in the flank of athymic mice following the same strategic approach described above. After three weeks of growth the tumours were injected with 2.5×10⁹ plaque-forming units (pfu) of Ad/sgBA1-Cas9 or PBS four times at days 16, 19, 22 and 24. Adenoviral delivery of both targeting sgRNAs and the Cas9 nuclease led to significant tumour growth inhibition, resulting in an average tumour size of 128.77 (+63.53) mm³ (P<0.05). Tumour volumes in control groups treated with PBS increased over time and reached an average size of 1853.91 mm³, 6 days after treatment (FIGS. 12a and b). Importantly, mice treated with Ad/sgBA1Cas9 showed no associated mortality in the 60 days of the study, whereas all controls needed to be sacrificed after two weeks of cell injection.

Robust CRISPR-Cas13-Mediated Knockdown of EWSR1-FLI1 and BCR-ABL1 mRNA Expression in ES and CML Cancer Cell Lines

To achieve the highest possible silencing of EWSR1-FLI1 and BCR-ABL1 mRNAs using the CRISPR-Cas13 system, the Cas13 protein and crRNAs are expressed from a lentiviral vector, LV-Cas13-crRNA. A series of crRNAs are tested to choose the most efficient (a representative example is shown in Table 5). The guide fragments in the series of crRNAs cover all of the positions containing the EWSR1-FLI1 and BCR-ABL1 breakpoints. The analysis of EWSR1-FLI1 or BCR-ABL1 mRNA expression levels of Cas13-crRNA lentivirus transduced ES or CML cancer cells show a decrease after transduction. The crRNAs producing the highest EWSR1-FLI1 or BCR-ABL1 mRNA knockdown are chosen for subsequent experiments.

TABLE 5
crRNAs

> crRNA DNAJB1-PRKACA
SEQ ID NO: 144
GCTACGGGGAGGAAGTGAAAGAATTCTTAG
> crRNA EML4-ALK
SEQ ID NO: 145
GGAAAGGACCTAAAGTGTACCGCCGGAAGC
> crRNA PAX3-FOXO1
SEQ ID NO: 146
GGCAGTATGGACAAAAATTCAATTCGTCAT
> crRNA TPM3-NTRK1
SEQ ID NO: 147
GATAAACTCAAGGAGACACTAACAGCACAT

EWSR1-FLI1 and BCR-ABL1 Knockdown by CRISPR-Cas13 is Highly Specific and Blocks the Proliferation of ES and CML Cancer Cell

The antitumor effects of CRISPR-Cas13-mediated EWSR1-FLI1 and BCR-ABL mRNA knockdown is evaluated in ES and CML cancer cells. First, we confirm that LV transduction of Cas13 and crRNA significantly reduces EWSR1-FLI1 and BCR-ABL mRNA expression levels in a time-dependent manner. Long-term and soft agar cell culture together with subG1 assay are used to measure the cell growth rate and apoptosis levels in cells treated with the CRISPRCas13 system. Transduction with the LV-Cas13-crRNA vector significantly suppress the growth and increase apoptotic cell rates of ES and CIVIL cancer cells. There is no obvious change in the growth or apoptosis rates in control cells after treatment with the CRISPR-Cas13 system.

KRAS-g12d Knockdown with CRISPR-Cas13 Inhibits Tumour Growth In Vivo.

To explore the antitumor potency of the CRISPR-Cas13 system in vivo, mice bearing subcutaneous ES or CML xenografts are treated with repeated intratumoural injections of the optimized CRISPR-Cas13 system delivered by adenoviral vectors. Compared to the control group, the Cas13-crRNA treated tumours group shows a significant volume reduction. The antitumour efficacy of the EWSR1-FLI1 or BCR-ABL1 knockdown approach is further investigated by histological and immunohistochemical analysis. H&E staining reveals a markedly reduce number of viable tumour cells and more extensive necrotic regions in treated cells than in controls. Moreover, CRISPR edited cells exhibit lower levels of KI67 proliferation marker compared with control tumours. Fusion mRNA knockdown tumours show an increase in caspase-3 protein expression compared with control tumours. Importantly, mice treated with Ad/Cas13-crRNA show lower mortality during the study.

Elimination of Cancer Cells Comprising Genomic Amplifications

Efficient NHEJ CRISPR-mediated genome editing strategy for targeting amplifications was achieved. The approach was based on targeting an intronic sequence of the amplified gene to induce multiple DNA breaks so much as copies of the gene are present in the amplified region. The gene editing-based approach only induced the deletion and damage in cells harbouring a gene amplification without affecting exonic sequences or protein expression of the germline non-amplified cells (FIG. 13).

Neuroblastoma

A cellular model of neuroblastoma, the most common extracranial solid tumor of childhood, in which MYCN is found amplified in 25% of the cases and correlates with high-risk disease and poor prognosis, was used. The first intron of MYCN gene was targeted.

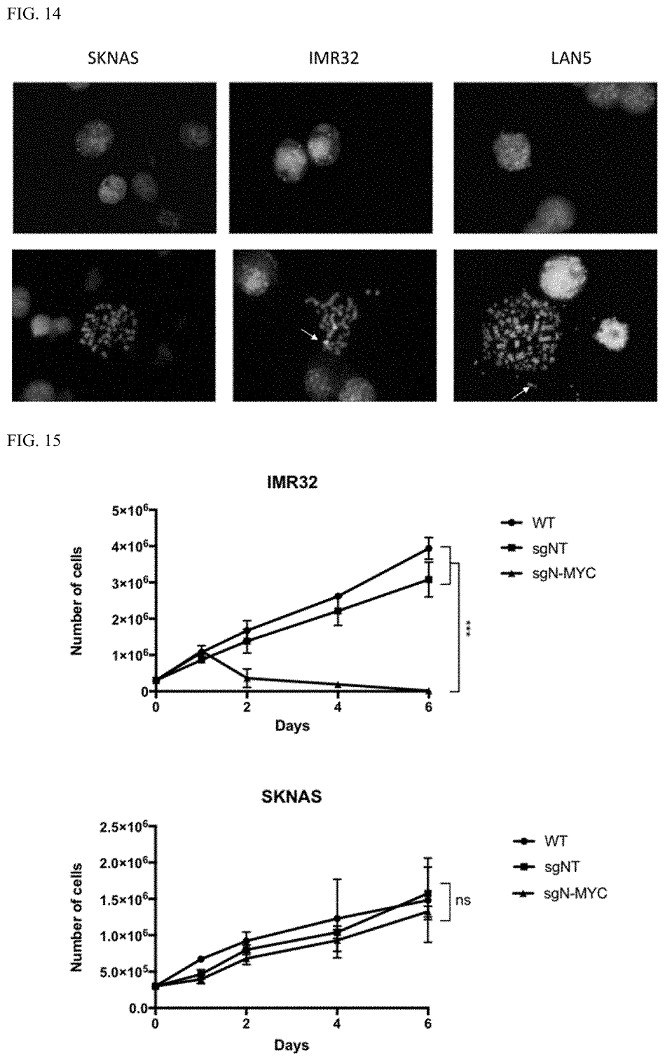

Neuroblastoma cell lines SKNAS, IMR32 and LANS were characterized to determine the type of amplification using FISH analysis with a MYCN probe to detect MYCN amplification. FISH analysis showed the presence of homology staining region (HSR) MYCN amplification in IMR32 cell line and double minutes-based amplification in LANS, whereas SKNAS does not harbor any MYCN amplification and was used as negative control (FIG. 14).

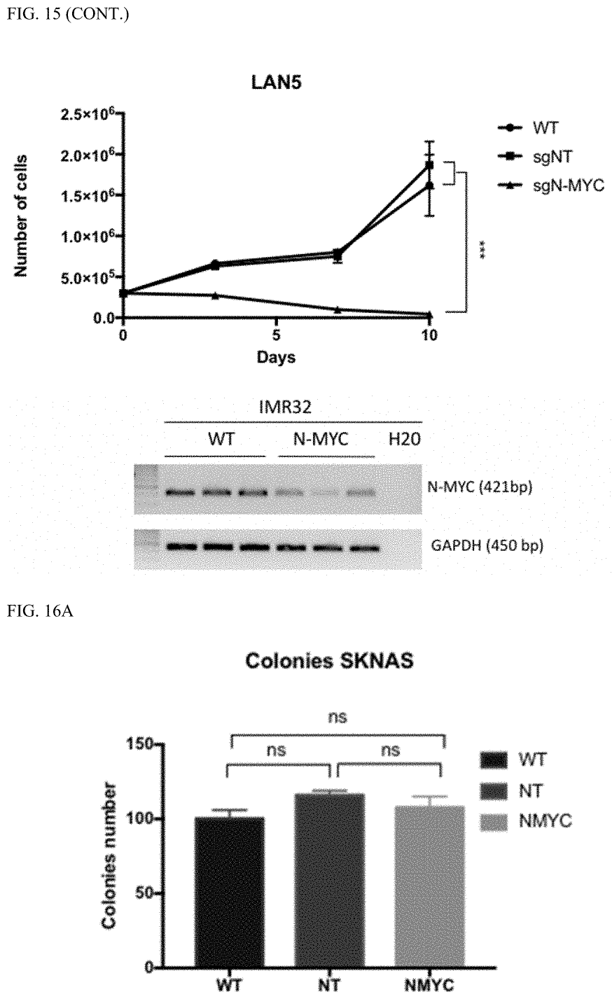

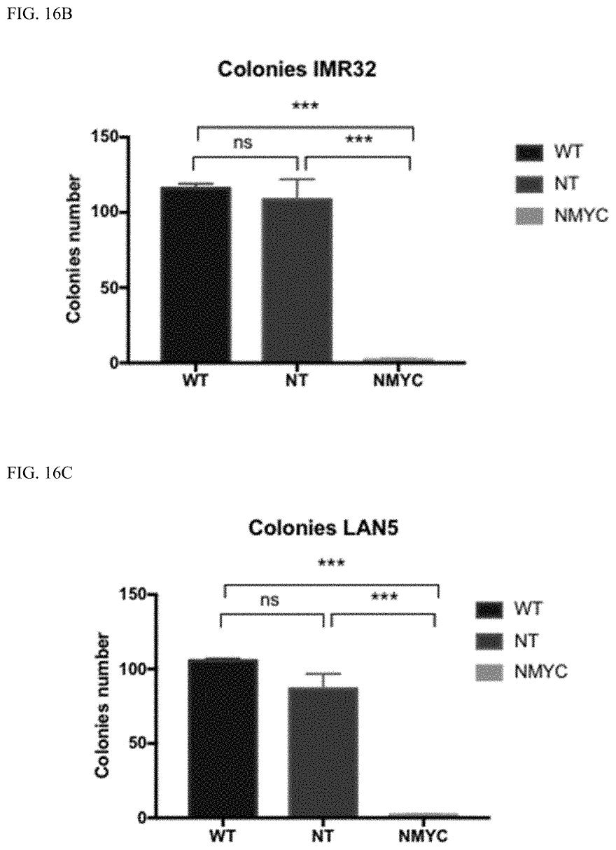

In vitro assays were performed to examine the functional consequences of targeting an intronic region of MYCN. Transduction with a guide targeting MYCN (sgMYCN), but not with a non targeting guide (sgNT), resulted in a robust decrease in IMR32 and LANS growth (FIG. 15) and clonogenic capacity in colony assays (FIG. 16), whereas these parameters were unchanged in control SKNAS cells (non-MYCN amplified) (FIGS. 15 and 16). Consistent with these observations, the growth phenotype was accompanied by death of the amplified cell lines (FIG. 15) and decreased levels of MYCN RNA levels (FIG. 15) when treated with sgMYCN.

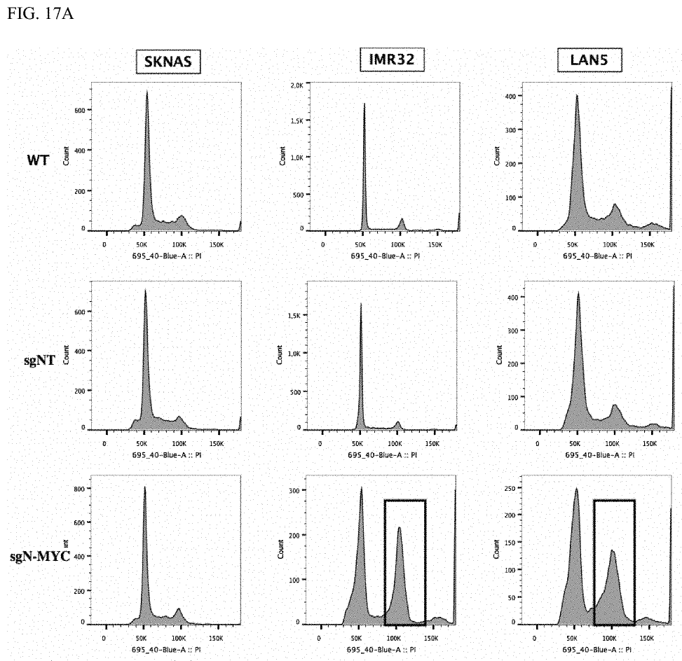

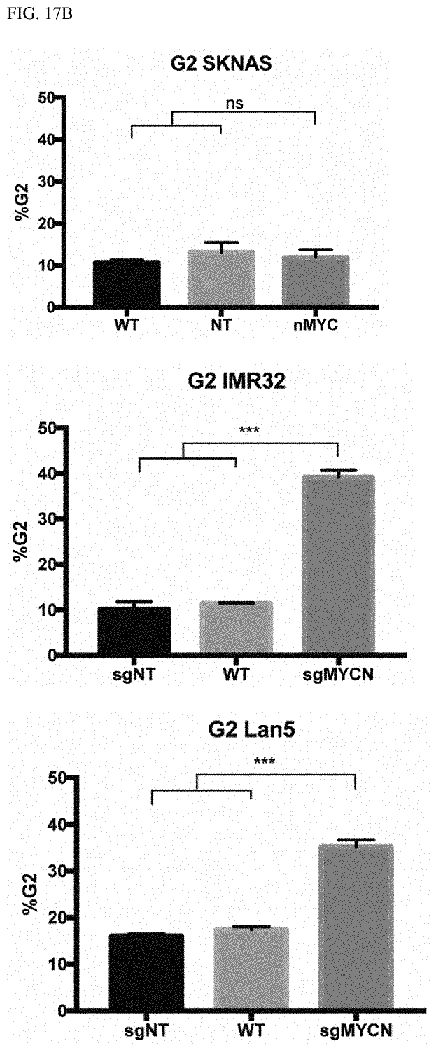

Also consistent with these observations, it was observed that when treated with sgMYCN IMR32 and LANS cells were arrested in G2 cell cycle phase, measured with propidium iodide staining, whereas normal cell cycle progression was observed in SKNAS (FIG. 17).

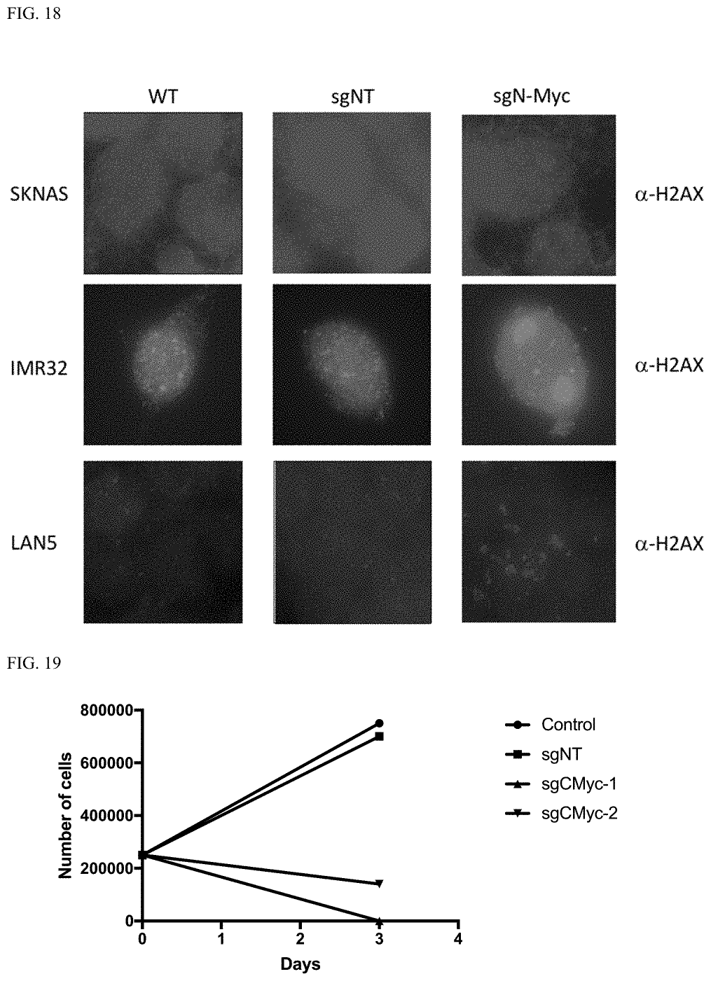

Immunofluorescence anti-H2AX showed an increase in the DNA repair foci in MYCN amplified cell lines after treatment with sgMYCN (FIG. 18).

Medulloblastoma

Another model was used: a cellular model of medulloblastoma, the most common cancerous brain tumor in children, in which MYC amplification oncogene is present in about 50% of high-risk neuroblastomas and correlates with high-risk disease and poor prognosis. Intron one of MYC gene was targeted to produce deletions and damage in medulloblastoma cell lines with MYC amplified and to guarantee the germline of cells without amplification. Medulloblastoma cell line MDB-HTB-185 was used. In vitro assays were performed to examine the functional consequences of targeting this intronic region of MYC.

Two sequences were designed: sgMYC-1 (SEQ ID NO: 152) and sgMYC-2 (SEQ ID NO: 153). sgRNAs were cloned in LentiCRIPSRv2 plasmid. Transduction with LVCas9 MYC-1 and LVCas9 MYC-2, but not with LVCas9 NT, resulted in a robust MDB-HTB-185 cell death (FIG. 19).

Other examples of cancers are assayed using gRNAs directed to noncoding and non regulatory genomic regions of oncogenes present in amplicons described in said cancers. sgRNAs are cloned in LentiCRIPSRv2 plasmid, which are transduced into a cell line of the corresponding cancer. They are the following:

| Cancer | Gene | sgRNA sequence |
|---|---|---|
| Rhabdomyosarcoma | FOXO1 | SEQ ID NO: 154 |
| Colon | MYC | SEQ ID NO: 152 |
| Mama | ERBB2 (Her2) | SEQ ID NO: 155 |
| Glioblastoma | EGFR | SEQ ID NO: 156 |
| Lung | MET | SEQ ID NO: 157 |
| Gastric | FGFR2 | SEQ ID NO: 158 |
| Oral squamous carcinoma | CCND1 | SEQ ID NO: 159 |
| Osteosarcoma | MDM2 | SEQ ID NO: 160 |
| Ovarian | RAB25 | SEQ ID NO: 161 |
| Retinoblastoma | MDM4 | SEQ ID NO: 162 |
| Testicular germ cell tumour | KRAS | SEQ ID NO: 163 |
| Bladder | AURKA | SEQ ID NO: 164 |
| Adrenocortical carcinoma | TERT | SEQ ID NO: 165 |

sgRNA Design and Generation of Lentiviral Constructs

sgRNAs were designed using the online Benchling CRISPR gRNA Design tool (http://www.benchling.com). The sgRNAs chosen were based on a high specificity rank and a low potential off-target effect³⁷. sgRNAs were cloned in LentiCRIPSRv2 plasmid. The sequences for sgRNAs used are for sgMYCN: CGGTCGCAATCTGGGTCACG (SEQ ID NO: 148) and sgNT: CCGCGCCGTTAGGGAACGAG (SEQ ID NO: 149); sgMYC-1: CATCTCCGTATTGAGTGCGA (SEQ ID NO: 152), sgMYC-2: CCCGTTAACATTTTAATTGC and sgNT: CCGCGCCGTTAGGGAACGAG (SEQ ID NO: 153).

qRT-PCR Analysis

RT-PCR amplification were performed using Q5 Taq DNA Polymerase (NEB). qRT-PCR was performed in 96-well plates with 2×SYBR Green Master Mix (ThermoFisher Sci) using an ABI-Prism7900HT Detection System (ThermoFisher Sci). Expression levels were normalised to the housekeeping gene GAPDH. The primers used were: RT-MYCN-fw: GAGACACCCGCGCAGAATC (SEQ ID NO: 150) and RT-MYCN-RV: CGTTCTCAAGCAGCATCTCC (SEQ ID NO: 151).

Cell Culture

IMR32, LANS and SKNAS were a gift from Dra Africa Gonzalez Murillo (Hospital Nino Jesús, Madrid). IMR32 and LANS cells were maintained in Roswell Park Memorial Institute medium (Gibco); and SKNAS were maintained in Dulbecco's modified Eagle's medium (Lonza) both were supplemented with 1% Glutamax (Life Technologies), 10 mg/ml antibiotics (penicillin and streptomycin) (Gibco) and 10% fetal bovine serum (FBS) (Life Technologies). All cells were cultured at 37° C. at 5% CO₂, 5% O₂ atmosphere in a humidified incubator. All cell lines used in this study were negative for mycoplasma contamination.

MDB-HTB-185 cell line were maintained in Alpha MEM medium (Gibco) supplemented with 1% Glutamax (Life Technologies), 10 mg/ml antibiotics (penicillin and streptomycin) (Gibco) and 10% fetal bovine serum (FBS) (Life Technologies). Cells were cultured at 37° C. at 5% CO₂, 5% O₂ atmosphere in a humidified incubator. All cell lines used in this study were negative for mycoplasma contamination.

Immunoassays

To detect DNA repair foci, transduced cells were seeded onto glass coverslips coated with poly-L-lysine (Cultek). After 72 h, cells were washed twice with d-PBS (Sigma), fixed in 4% paraformaldehyde (PFA; Electron Microscope Sci) for 12 min at room temperature (RT), permeabilised with 0.3% Triton X-100 (Sigma) in PBS and blocked with 3% normal goat serum (NGS; Sigma) in PBS for 1 h at RT. Thereafter, samples were incubated overnight at 4° C. with an anti-H2AX antibody (1/500; SIGMA) diluted in PBS supplemented with 1% NGS, and then with an Alexa Fluor-594-conjugated secondary antibody (1/500; ThermoFisher Sci) for 1 h at RT. Finally, samples were counterstained with DAPI (Vecotor Labs), air dried and mounted in Vectashield mounting medium (Vector Labs). Images were acquired on a Leica DM5500B microscope with two lasers with excitation at 594 nm (red channel, H2AX detection) and 405 nm (blue channel, nuclear DAPI staining). Data were collected sequentially at a resolution of 1024×1024 pixels and are representative of every experiment carried out using a Cytovision v7.4 software (Leica Biosystem).

Fluorescence In Situ Hybridization (FISH)

The MYCN amplification FISH probe (Vysis) was used to detect MYCN chromosomal amplification. 5 mm tissue sections were deparaffined in xylene and rehydrated in ethanol. Tissue sections were pre-treated in 2-[N-morpholino]ethanesulphonic acid (MES, DAKO), followed by pepsin digestion (DAKO). After dehydration, the samples were denatured in the presence of the EWSR1/FLI1 probe at 66° C. for 10 min and left overnight for hybridization at 37° C. in a hybridizer machine (DAKO). Then, the slides were washed with 20×SSC-Tween20 buffer at 63° C. and mounted on fluorescence mounting medium (DAPI). FISH signals were manually scored by counting the number of nuclei with dual-fusion signals all over the tissue. FISH images were captured using a CCD camera (Photometrics SenSys camera) connected to a PC running the Zytovision image analysis system (Applied Imaging Ltd., UK).

### BIBLIOGRAPHY

1. Rabbitts T H. Nature 372, 143-149 (1994). DOI: 10.1038/372143a0.
2. Mitelman F et al. Mitelman database of chromosome aberrations and gene fusions in cancer. NCI [online] (2017).
3. Mitelman F et al. Nat Rev Cancer 2007; 7(4):233-245.
4. Licht J D. N Engl J Med 2009; 360 (9):928-930.
5. Druker B J et al. N Engl J Med 2001; 344 (14):1031-1037.
6. Borden E C et al. Clin. Cancer Res. 9, 1941-1956 (2003). PMID:12796356
7. Johansson B et al. Ann. Med. 36, 492-503 (2004). DOI:10.1080/07853890410018808
8. Mrozek K et al. Blood Rev. 18, 115-36(2004). DOI:10.1016/S0268-960X(03)00040-7
9. Mitelman F et al. Gen Chrom & Cancer 43, 350-66 (2005). DOI:10.1002/gcc.20212
10. Antonescu C R. Histopathol 48, 13-21(2006). DOI:10.1111/j.1365-2559.2005.02285.x
11. Oehler V G & Radich J P. Curr Oncol Rep. 5, 426-435 (2003). PMID:12895396
12. Avigad S et al. Cancer 100, 1053-1058 (2004). DOI:10.1002/cncr.20059
13. Deininger M et al. Blood 105, 2640-2653 (2005). DOI:10.1182/blood-2004-08-3097
14. Kern W et al. Crit Rev Oncol Hematol. 56, 283-309 (2005). DOI:10.1016/j.critrevonc.2004.06.004
15. Rodriguez-Garcia A et al. Curr Cancer Drug Targets 1, 109-19(2001). PMID:12188884
16. Thomas et al. Act Pharmcol 27, 273-81 (2006) DOI:10.1111/j.1745-7254.2006.00282.x
17. Mojica et al. J Mol Evol 60, 174-182 (2005). DOI:10.1007/s00239-004-0046-3
18. Jinek et al. Science 337, 816-821 (2012). DOI:10.1126/science.1225829
19. Esvelt et al. eLife 3, 03401 (2014). DOI:10.7554/eLife.03401
20. Chen et al. Nat Biotech 35 (6) 543-550 (2017). DOI:10.1038/nbt.3843
21. P. Mohanraju, et al. Science 353 (2016) aad5147.
22. O. O. Abudayyeh, et al. Science 353 (2016) aaf5573.
23. J. S. Gootenberg, et al. Science 356 (2017) 438-442.
24. L. Liu, et al. Cell 170 (2017) 714-726 e710.
25. L. Liu, et al. Cell 168 (2017) 121-134 e112.
26. A. East-Seletsky, et al. Mol. Cell 66 (2017) 373-383 e373.
27. A. East-Seletsky, et al. Nature 538 (2016) 270-273.
28. D. B. T. Cox, et al. Science 358 (2017) 1019-1027.
29. O. O. Abudayyeh, et al. Nature 550 (2017) 280-284.
30. X. Zhao et al. Cancer Letters 431 (2018) 171-181.
31. Santarius et al (2010) Nature Reviews Cancer volume 10, 59-64.
32. Albertson D G (2006) Trends Genet 22: 447-455.
33. Schwab M. Bioessays 1998: 20(6):473-479.
34. Chen et al (2014) PLoS One. 2014; 9(5): e98293
35. Matsui et al (2013) Biomol Concepts. 2013 December; 4(6):567-82.
36. Myllykangas et al. 2007 Semin Cancer Biol 17: 42-55.
37. Doench N. F. et al. Nat Biotechnol. 2016 February; 34(2): 184-191.
38. Albertson D G et al. Nat Genet. 2003 August; 34(4):369-76. Review.

1. A method for eliminating cancer cells, wherein said cells comprise a genomic rearrangement which leads to the expression of a fusion gene not present in non-cancer cells, said method comprising
a. cleaving the genome in at least two sites, said cleavage leading to either a deletion, an inversion, a frameshift, the cleavage without repair and/or an insertion in the genome of said cancer cells, and/or
b. cleaving the expression product of said fusion gene or cancer inducing gene in at least one site.

2. The method according to claim 1, wherein the cancer cells comprise a genomic rearrangement which leads to the expression of the rearranged gene not present in non-cancer cells, preferably leads to the expression of a fusion gene not present in non-cancer cells.

3. The method according to claim 1, wherein cleaving the genome leads to a deletion, an inversion, a frameshift, the cleavage without repair or any combination thereof.

4. The method according to claim 1, wherein said method comprises cleaving the genome in two sites, or in three sites or in four sites.

5. The method according to claim 1, wherein the genomic rearrangement leads to the expression of a fusion gene selected from EWSR1-FLI1, BCR-ABL, DNAJB1-PRKACA, EML4-ALK, PAX3-FOXO1 and TPM3-NTRK1, preferably leads to the expression of fusion gene EWSR1-FLI1 or BCR-ABL.

6. (canceled)

7. (canceled)

8. The method according to claim 1, wherein the cleavage is in a genomic region other than a coding region or a regulatory region, preferably the cleavage is in an intronic region, more preferably the cleavage is in an intronic region of a genomic amplification other than the splice sites.

9. (canceled)

10. (canceled)

11. The method according to claim 1, wherein the cleaving is done by an endonuclease selected from a CRISPR associated protein, a zinc-finger nuclease (ZFN) and a transcription activator-like effector nuclease (TALEN).

12. The method according to claim 1, wherein the cleaving is done by a Cas protein, preferably Cas9 or Cas13, more preferably Cas9.

13. The method according to claim 1, wherein at least one guide RNA (gRNA) is used to target the cleaving of the genome, preferably at least two gRNAs are used to target the cleaving of the genome.

14. The method according to claim 1, wherein the target of said endonuclease is in an intron of a fusion gene present in cancer cells and absent in non-cancer cells and wherein said target is not patient-specific.

15. A kit of parts comprising at least two endonucleases, preferably selected from a zinc-finger nuclease (ZFN) and a transcription activator-like effector nuclease (TALEN), wherein said endonucleases specifically cleave the genome in at least two sites and wherein said cleavage leads to either a deletion, a frameshift and/or an insertion in the genome, preferably a deletion and/or a frameshift; or
comprising
(a) a CRISPR associated endonuclease, preferably a Cas protein, more preferably Cas9 or Cas13, more preferably a Cas9; and
(b) at least two gRNAs that have a targeting domain in a genomic rearrangement present in a cancer cell which leads either to the expression a fusion gene not present in non-cancer cells or to rearrangements which lead to the induction of the expression or the overexpression of a cancer inducing gene.

16. (canceled)

17. (canceled)

18. The kit of parts according to claim 15 comprising:
a. the nuclease with amino acid sequence SEQ ID NO: 1; and
b. the pair of gRNAs with nucleotide sequences SEQ ID NO: 2 and SEQ ID NO: 3; or the pair of gRNAs with nucleotide sequences SEQ ID NO: 4 and SEQ ID NO: 5; or a pair of gRNAs with nucleotide sequences SEQ ID NO: 128 or SEQ ID NO: 129 and SEQ ID NO: 130 or SEQ ID NO: 131; or a pair of gRNAs with nucleotide sequences SEQ ID NO: 132 or SEQ ID NO: 133 and SEQ ID NO: 134 or SEQ ID NO: 135; or a pair of gRNAs with nucleotide sequences SEQ ID NO: 136 or SEQ ID NO: 137 and SEQ ID NO: 138 or SEQ ID NO: 139; or a pair of gRNAs with nucleotide sequences SEQ ID NO: 140 or SEQ ID NO: 141 and SEQ ID NO: 142 or SEQ ID NO: 143.

19. (canceled)

20. (canceled)

21. (canceled)

22. A nucleic acid comprising the codifying sequence for:
a. a CRISPR associated endonuclease, preferably a Cas protein, more preferably Cas9 or Cas13, even more preferably Cas9;
b. at least one gRNA that has a targeting domain in the expression product of a fusion gene or at least a pair of gRNAs that have a targeting domain in a genomic rearrangement present in a cancer cell which leads to the expression of a fusion gene not present in non-cancer cells.

23. (canceled)

24. A nucleic acid according to claim 22 comprising the codifying sequence for:
a. the nuclease with amino acid sequence SEQ ID NO: 1; and
b. the pair of gRNAs with nucleotide sequences SEQ ID NO: 2 and SEQ ID NO: 3; or the pair of gRNAs with nucleotide sequences SEQ ID NO: 4 and SEQ ID NO: 5; or a pair of gRNAs with nucleotide sequences SEQ ID NO: 128 or SEQ ID NO: 129 and SEQ ID NO: 130 or SEQ ID NO: 131; or a pair of gRNAs with nucleotide sequences SEQ ID NO: 132 or SEQ ID NO: 133 and SEQ ID NO: 134 or SEQ ID NO: 135; or a pair of gRNAs with nucleotide sequences SEQ ID NO: 136 or SEQ ID NO: 137 and SEQ ID NO: 138 or SEQ ID NO: 139; or a pair of gRNAs with nucleotide sequences SEQ ID NO: 140 or SEQ ID NO: 141 and SEQ ID NO: 142 or SEQ ID NO: 143.

25. (canceled)

26. (canceled)

27. (canceled)

28. A method for treating a subject afflicted from fibrolamellar hepatocellular carcinoma, non-small cell lung cancer, alveolar rhabdomyosarcoma, glioblastoma, colorectal cancer, acute lymphocytic leukemia, Ewing sarcoma, bladder cancer, neuroblastoma, medulloblastoma, breast cancer, gastric cancer, oral squamous carcinoma, osteosarcoma, ovarian cancer, retinoblastoma, testicular germ cell tumor or adrenocortical carcinoma comprising the method of eliminating cancer cells of claim 1.

29. A method for treating a subject afflicted from cancer comprising the method of eliminating cancer cells of claim 1.


SEQUENCE LISTING (165 sequences)

SEQ ID NO: 1 - protein, 1396 aa, Streptococcus pyogenes
MPKKKRKVGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 2 - DNA, 23 bp, Homo sapiens
tggttgcacagtaagtggcgggg

SEQ ID NO: 3 - DNA, 23 bp, Homo sapiens
agtgggccacactgcgacaaggg

SEQ ID NO: 4 - DNA, 20 bp, Homo sapiens
tatccgaggcacgttaaggg

SEQ ID NO: 5 - DNA, 20 bp, Homo sapiens
cacgaggttgacgcaccaga

SEQ ID NO: 6 - protein, 504 aa, Homo sapiens
MASTDYSTYSQAAAQQGYSAYTAQPTQGYAQTTQAYGQQSYGTYGQPTDVSYTQAQTTATYGQTAYATSYGQPPTVEGTSTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQNPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 7 - protein, 68 aa, Homo sapiens
MASTDYSTYSQAAAQQGYSAYTAQPTQGYAQTTQEAGRSSCGNSSWSCSPTAPTPAVSPGRGPTGSSK

SEQ ID NO: 8 - DNA, 81 bp, Homo sapiens
gcagtgcatagatattaagtaacttgccagtggttgcacagtaagtggcggggttagctctaaaaactggcgacctagcca

SEQ ID NO: 9 - DNA, 81 bp, Homo sapiens
gcagtgcatagatattaagtaacttgccagtggttgcacagtaagtgcggggttagctctaaaaactggcgacctagccaa

SEQ ID NO: 10 - DNA, 84 bp, Homo sapiens
gcagtgcatagatattaagtaacttgccagtggttgcacagtaagtgtggcggggttagctctaaaaactggcgacctagccaa

SEQ ID NO: 11 - DNA, 83 bp, Homo sapiens
gcagtgcatagatattaagtaacttgccagtggttgcacagtaagtgggcggggttagctctaaaaactggcgacctagccaa

SEQ ID NO: 12 - DNA, 79 bp, Homo sapiens
gcagtgcatagatattaagtaacttgccagtggttgcacagtaagcggggttagctctaaaaactggcgacctagccaa

SEQ ID NO: 13 - DNA, 70 bp, Homo sapiens
gcagtgcatagatattaagtaacttgccagtggttgcggggttagctctaaaaactggcgacctagccaa

SEQ ID NO: 14 - DNA, 80 bp, Homo sapiens
gcagtgcatagatattaagtaacttgccagtggttgcacagtaaggcggggttagctctaaaaactggcgacctagccaa

SEQ ID NO: 15 - DNA, 67 bp, Homo sapiens
gcagtgcatagatattaagtaacttgccagtggcggggttagctctaaaaactggcgacctagccaa

SEQ ID NO: 16 - DNA, 75 bp, Homo sapiens
gcagtgcatagatattaagtaacttgccagtggttgcacagcggggttagctctaaaaactggcgacctagccaa

SEQ ID NO: 17 - DNA, 70 bp, Homo sapiens
gcagtgcatagatattaagtaacttgccagtggttgcacagttagctctaaaaactggcgacctagccaa

SEQ ID NO: 18 - DNA, 62 bp, Homo sapiens
gcagtgcatagatattaagtaacttgccagtggttagctctaaaaactggcgacctagccaa

SEQ ID NO: 19 - DNA, 78 bp, Homo sapiens
gcagtgcatagatattaagtaacttgccagtggttgcacagtagcggggttagctctaaaaactggcgacctagccaa

SEQ ID NO: 20 - DNA, 83 bp, Homo sapiens
tgggtaggcagagtccctgggatgggaaggtgagtgggccacactgcgacaagggcctgctagctcccaatctcgatggactt

SEQ ID NO: 21 - DNA, 84 bp, Homo sapiens
tgggtaggcagagtccctgggatgggaaggtgagtgggccacactgcgaacaagggcctgctagctcccaatctcgatggactt

SEQ ID NO: 22 - DNA, 70 bp, Homo sapiens
tgggtaggcagagtccctgggatgggaaggtgagtgggccacactgctagctcccaatctcgatggactt

SEQ ID NO: 23 - DNA, 66 bp, Homo sapiens
tgggtaggcagagtccctgggatgggaaggtgagtgggcctgctagctcccaatctcgatggactt

SEQ ID NO: 24 - DNA, 82 bp, Homo sapiens
tgggtaggcagagtccctgggatgggaaggtgagtgggccacactgcgaaagggcctgctagctcccaatctcgatggactt

SEQ ID NO: 25 - DNA, 80 bp, Homo sapiens
tgggtaggcagagtccctgggatgggaaggtgagtgggccacactgcgagggcctgctagctcccaatctcgatggactt

SEQ ID NO: 26 - DNA, 80 bp, Homo sapiens
tgggtaggcagagtccctgggatgggaaggtgagtgggccacactgcaagggcctgctagctcccaatctcgatggactt

SEQ ID NO: 27 - DNA, 81 bp, Homo sapiens
tgggtaggcagagtccctgggatgggaaggtgagtgggccacactgcgaagggcctgctagctcccaatctcgatggactt

SEQ ID NO: 28 - DNA, 74 bp, Homo sapiens
tgggtaggcagagtccctgggatgggaaggtgagtgggccacactgcgagctagctcccaatctcgatggactt

SEQ ID NO: 29 - DNA, 65 bp, Homo sapiens
tgggtaggcagagtccctgggatgggaaggtgagtgggccacactgctcccaatctcgatggact

SEQ ID NO: 30 - DNA, 82 bp, Homo sapiens
tgggtaggcagagtccctgggatgggaaggtgagtgggccacactgcgcaagggcctgctagctcccaatctcgatggactt

SEQ ID NO: 31 - DNA, 75 bp, Homo sapiens
tgggtaggcagagtccctgggatgggaaggtgagtgggccacaagggcctgctagctcccaatctcgatggactt

SEQ ID NO: 32 - DNA, 5194 bp, Streptococcus pyogenes
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggttgcacagtaagtggcggtttaagagctatgctggaaacagcatagcaagtttaaataaggctagtccgttatcaacttgaaaaagtggcaccgagtcggtgctttttgctagccgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccggtgggccacactgcgacaagtttaagagctatgctggaaacagcatagcaagtttaaataaggctagtccgttatcaacttgaaaaagtggcaccgagtcggtgctttttctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaatag

SEQ ID NO: 33 - DNA, 23 bp, Homo sapiens
taagaggacatacagcgttctgg

SEQ ID NO: 34 - DNA, 23 bp, Homo sapiens
atttggacctgtggcgatatggg

SEQ ID NO: 35 - DNA, 23 bp, Homo sapiens
cgggcggatcatataaggtcagg

SEQ ID NO: 36 - DNA, 23 bp, Homo sapiens
caagtcgatcccaatgtcgaagg

SEQ ID NO: 37 - DNA, 23 bp, Homo sapiens
tagatcgcgtactccatcctggg

SEQ ID NO: 38 - DNA, 20 bp, Homo sapiens
gacatgaccatgggtaagcg

SEQ ID NO: 39 - DNA, 20 bp, Homo sapiens
tccctaatagtgatggcgct

SEQ ID NO: 40 - DNA, 20 bp, Homo sapiens
gcccagcccactcaaggata

SEQ ID NO: 41 - DNA, 20 bp, Homo sapiens
ttggggttggggtagattcc

SEQ ID NO: 42 - DNA, 19 bp, Homo sapiens
gcagggctacagtgcttac

SEQ ID NO: 43 - DNA, 19 bp, Homo sapiens
gcagctccaggaggaattg

SEQ ID NO: 44 - DNA, 19 bp, Homo sapiens
aggtgccctgttccatgct

SEQ ID NO: 45 - DNA, 19 bp, Homo sapiens
aggtgccctgttccatgct

SEQ ID NO: 46 - DNA, 20 bp, Homo sapiens
gtgagtttaccttggcctgc

SEQ ID NO: 47 - DNA, 20 bp, Homo sapiens
gtgagtttaccttggcctgc

SEQ ID NO: 48 - DNA, 20 bp, Homo sapiens
cccagccagatccgtatcag

SEQ ID NO: 49 - DNA, 19 bp, Homo sapiens
gcagctccaggaggaattg

SEQ ID NO: 50 - DNA, 19 bp, Homo sapiens
gcagggctacagtgcttac

SEQ ID NO: 51 - DNA, 23 bp, Homo sapiens
aagacgtcttgctcccctcgggg

SEQ ID NO: 52 - DNA, 19 bp, Homo sapiens
accacagtccatgccatca

SEQ ID NO: 53 - DNA, 20 bp, Homo sapiens
tccaccaccctgttgctgta

SEQ ID NO: 54 - DNA, 20 bp, Homo sapiens
gatgccaaggatccaacgac

SEQ ID NO: 55 - DNA, 20 bp, Homo sapiens
ggcttcacaccattccccat

SEQ ID NO: 56 - DNA, 22 bp, Homo sapiens
gctgtcatctcttgtgggctgt

SEQ ID NO: 57 - DNA, 21 bp, Homo sapiens
actcatgggagctgctggttc

SEQ ID NO: 58 - DNA, 19 bp, Homo sapiens
accacagtccatgccatca

SEQ ID NO: 59 - DNA, 20 bp, Homo sapiens
tccaccaccctgttgctgta

SEQ ID NO: 60 - DNA, 47 bp, Homo sapiens
ctttccctacacgacgctcttccgatctaggtgccctgttccatgct

SEQ ID NO: 61 - DNA, 53 bp, Homo sapiens
gactggagttcagacgtgtgctcttccgatcttggcctaggcttttcaacaga

SEQ ID NO: 62 - DNA, 48 bp, Homo sapiens
ctttccctacacgacgctcttccgatctatggtattctcacgctgcca

SEQ ID NO: 63 - DNA, 53 bp, Homo sapiens
gactggagttcagacgtgtgctcttccgatcttggagatgaatgggaagcgaa

SEQ ID NO: 64 - DNA, 51 bp, Homo sapiens
ctttccctacacgacgctcttccgatcttcccacttgtttattcctctgtg

SEQ ID NO: 65 - DNA, 54 bp, Homo sapiens
gactggagttcagacgtgtgctcttccgatctattccagagaaggacattgcca

SEQ ID NO: 66 - DNA, 48 bp, Homo sapiens
ctttccctacacgacgctcttccgatcttgggagttctctaaggctgc

SEQ ID NO: 67 - DNA, 52 bp, Homo sapiens
gactggagttcagacgtgtgctcttccgatctgtgacttttcccaccgcctc

SEQ ID NO: 68 - DNA, 50 bp, Homo sapiens
ctttccctacacgacgctcttccgatctctccttttctcctcctgccagc

SEQ ID NO: 69 - DNA, 52 bp, Homo sapiens
gactggagttcagacgtgtgctcttccgatctgacttggatcttcaaccgcc

SEQ ID NO: 70 - DNA, 52 bp, Homo sapiens
ctttccctacacgacgctcttccgatctagcttgctattctttgagatgaac

SEQ ID NO: 71 - DNA, 53 bp, Homo sapiens
gactggagttcagacgtgtgctcttccgatcttgaataaaggccccgatgacc

SEQ ID NO: 72 - DNA, 48 bp, Homo sapiens
ctttccctacacgacgctcttccgatctcctgggttgtaactgtgggt

SEQ ID NO: 73 - DNA, 54 bp, Homo sapiens
gactggagttcagacgtgtgctcttccgatctttctgggagtcgtaggcttagt

SEQ ID NO: 74 - DNA, 48 bp, Homo sapiens
ctttccctacacgacgctcttccgatctctcgggcctgttccttcata

SEQ ID NO: 75 - DNA, 52 bp, Homo sapiens
gactggagttcagacgtgtgctcttccgatctccacctccagaagcccttag

SEQ ID NO: 76 - DNA, 49 bp, Homo sapiens
ctttccctacacgacgctcttccgatctgcaagaatttcaaggccccag

SEQ ID NO: 77 - DNA, 52 bp, Homo sapiens
gactggagttcagacgtgtgctcttccgatctagggatgacttgactgctga

SEQ ID NO: 78 - DNA, 48 bp, Homo sapiens
ctttccctacacgacgctcttccgatctgtcactcacctggctgcttc

SEQ ID NO: 79 - DNA, 55 bp, Homo sapiens
gactggagttcagacgtgtgctcttccgatctagcattcctcatttgattccaga

SEQ ID NO: 80 - DNA, 47 bp, Homo sapiens
ctttccctacacgacgctcttccgatctgttgtctcccgcatgccag

SEQ ID NO: 81 - DNA, 51 bp, Homo sapiens
gactggagttcagacgtgtgctcttccgatctggaatgggtaggcagagtc

SEQ ID NO: 82 - DNA, 50 bp, Homo sapiens
ctttccctacacgacgctcttccgatctagggagggtctaatctaggagc

SEQ ID NO: 83 - DNA, 53 bp, Homo sapiens
gactggagttcagacgtgtgctcttccgatctccctcttccccaccattttgt

SEQ ID NO: 84 - DNA, 49 bp, Homo sapiens
ctttccctacacgacgctcttccgatctgcatacagggcttctttcgtg

SEQ ID NO: 85 - DNA, 52 bp, Homo sapiens
gactggagttcagacgtgtgctcttccgatctcgttcttcctgtgccatcct

SEQ ID NO: 86 - DNA, 48 bp, Homo sapiens
ctttccctacacgacgctcttccgatcttgtgtggaggagggagtcaa

SEQ ID NO: 87 - DNA, 53 bp, Homo sapiens
gactggagttcagacgtgtgctcttccgatctggccttcagaactcatcaagg

SEQ ID NO: 88 - DNA, 48 bp, Homo sapiens
ctttccctacacgacgctcttccgatctatcctcacagagcattgcag

SEQ ID NO: 89 - DNA, 53 bp, Homo sapiens
gactggagttcagacgtgtgctcttccgatctgtactgattctggggcttgct

SEQ ID NO: 90 - DNA, 49 bp, Homo sapiens
ctttccctacacgacgctcttccgatctcgttggctgtgtgtctgtttc

SEQ ID NO: 91 - DNA, 52 bp, Homo sapiens
gactggagttcagacgtgtgctcttccgatctaggagtggggagtctttcgt

SEQ ID NO: 92 - DNA, 48 bp, Homo sapiens
ctttccctacacgacgctcttccgatctaggagaccgatggacagacg

SEQ ID NO: 93 - DNA, 52 bp, Homo sapiens
gactggagttcagacgtgtgctcttccgatctcctccctcctttcccctgac

SEQ ID NO: 94 - DNA, 50 bp, Homo sapiens
ctttccctacacgacgctcttccgatcttccataagttgactctggcagg

SEQ ID NO: 95 - DNA, 52 bp, Homo sapiens
gactggagttcagacgtgtgctcttccgatctagagtgccttggtcaaatgg

SEQ ID NO: 96 - DNA, 49 bp, Homo sapiens
ctttccctacacgacgctcttccgatctagtgttgggattacaggcgtg

SEQ ID NO: 97 - DNA, 53 bp, Homo sapiens
gactggagttcagacgtgtgctcttccgatctgcctgggaatttcactgtgcc

SEQ ID NO: 98 - DNA, 47 bp, Homo sapiens
ctttccctacacgacgctcttccgatctagttcccctctcctccctg

SEQ ID NO: 99 - DNA, 52 bp, Homo sapiens
gactggagttcagacgtgtgctcttccgatctcacttcccatggacagcttg

SEQ ID NO: 100 - DNA, 49 bp, Homo sapiens
ctttccctacacgacgctcttccgatctgaggggtaaaggattggagcc

SEQ ID NO: 101 - DNA, 52 bp, Homo sapiens
gactggagttcagacgtgtgctcttccgatctcggagagattgaagggagcg

SEQ ID NO: 102 - DNA, 28 bp, Homo sapiens
gtcataagaagggttctgctgcccgtag

SEQ ID NO: 103 - DNA, 28 bp, Homo sapiens
ggccagcagtgaactctgctgcccgtag

SEQ ID NO: 104 - DNA, 28 bp, Homo sapiens
ccgctgaagggcttttgaactctgctta

SEQ ID NO: 105 - DNA, 20 bp, Homo sapiens
tggttgcacagtaagtggcg

SEQ ID NO: 106 - DNA, 20 bp, Homo sapiens
tgattgcactgtaagtggcc

SEQ ID NO: 107 - DNA, 20 bp, Homo sapiens
taggtgctcagtaagtggct

SEQ ID NO: 108 - DNA, 20 bp, Homo sapiens
cgacttcacagtaagtggcg

SEQ ID NO: 109 - DNA, 20 bp, Homo sapiens
aggtagcgcagttagtggcg

SEQ ID NO: 110 - DNA, 20 bp, Homo sapiens
aggtagaacagtaagtggca

SEQ ID NO: 111 - DNA, 20 bp, Homo sapiens
tagttgttcagtaagtggca

SEQ ID NO: 112 - DNA, 20 bp, Homo sapiens
gggtagcagaggaagtggcg

SEQ ID NO: 113 - DNA, 20 bp, Homo sapiens
tggatgctgagtaagtggcc

SEQ ID NO: 114 - DNA, 20 bp, Homo sapiens
tgcttgcaaggtaagtggcc

SEQ ID NO: 115 - DNA, 20 bp, Homo sapiens
agtgggccacactgcgacaa

SEQ ID NO: 116 - DNA, 20 bp, Homo sapiens
gattggccacactgtgacaa

SEQ ID NO: 117 - DNA, 20 bp, Homo sapiens
ggcggggcacacagcgacaa

SEQ ID NO: 118 - DNA, 20 bp, Homo sapiens
agtgaggaacactgcggcaa

SEQ ID NO: 119 - DNA, 20 bp, Homo sapiens
tgtgggccaggctgcggcaa

SEQ ID NO: 120 - DNA, 20 bp, Homo sapiens
agtgggctagcctgcgacag

SEQ ID NO: 121 - DNA, 20 bp, Homo sapiens
agtggggctgtctgcgacaa

SEQ ID NO: 122 - DNA, 20 bp, Homo sapiens
tgtgaaccacactgtgacaa

SEQ ID NO: 123 - DNA, 20 bp, Homo sapiens
agtgtgctccactgtgacaa

SEQ ID NO: 124 - DNA, 20 bp, Homo sapiens
cgtgggccagcctgggacaa

SEQ ID NO: 125 - DNA, 20 bp, Homo sapiens
agagagccacactgagacag

SEQ ID NO: 126 - protein, 1094 aa, Prevotella buccalis
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQ

SEQ ID NO: 127 - DNA, 3285 bp, Prevotella buccalis
atgaacatccccgctctggtggaaaaccagaagaagtactttggcacctacagcgtgatggccatgctgaacgctcagaccgtgctggaccacatccagaaggtggccgatattgagggcgagcagaacgagaacaacgagaatctgtggtttcaccccgtgatgagccacctgtacaacgccaagaacggctacgacaagcagcccgagaaaaccatgttcatcatcgagcggctgcagagctacttcccattcctgaagatcatggccgagaaccagagagagtacagcaacggcaagtacaagcagaaccgcgtggaagtgaacagcaacgacatcttcgaggtgctgaagcgcgccttcggcgtgctgaagatgtacagggacctgaccaaccactacaagacctacgaggaaaagctgaacgacggctgcgagttcctgaccagcacagagcaacctctgagcggcatgatcaacaactactacacagtggccctgcggaacatgaacgagagatacggctacaagacagaggacctggccttcatccaggacaagcggttcaagttcgtgaaggacgcctacggcaagaaaaagtcccaagtgaataccggattcttcctgagcctgcaggactacaacggcgacacacagaagaagctgcacctgagcggagtgggaatcgccctgctgatctgcctgttcctggacaagcagtacatcaacatctttctgagcaggctgcccatcttctccagctacaatgcccagagcgaggaacggcggatcatcatcagatccttcggcatcaacagcatcaagctgcccaaggaccggatccacagcgagaagtccaacaagagcgtggccatggatatgctcaacgaagtgaagcggtgccccgacgagctgttcacaacactgtctgccgagaagcagtcccggttcagaatcatcagcgacgaccacaatgaagtgctgatgaagcggagcagcgacagattcgtgcctctgctgctgcagtatatcgattacggcaagctgttcgaccacatcaggttccacgtgaacatgggcaagctgagatacctgctgaaggccgacaagacctgcatcgacggccagaccagagtcagagtgatcgagcagcccctgaacggcttcggcagactggaagaggccgagacaatgcggaagcaagagaacggcaccttcggcaacagcggcatccggatcagagacttcgagaacatgaagcgggacgacgccaatcctgccaactatccctacatcgtggacacctacacacactacatcctggaaaacaacaaggtcgagatgtttatcaacgacaaagaggacagcgccccactgctgcccgtgatcgaggatgatagatacgtggtcaagacaatccccagctgccggatgagcaccctggaaattccagccatggccttccacatgtttctgttcggcagcaagaaaaccgagaagctgatcgtggacgtgcacaaccggtacaagagactgttccaggccatgcagaaagaagaagtgaccgccgagaatatcgccagcttcggaatcgccgagagcgacctgcctcagaagatcctggatctgatcagcggcaatgcccacggcaaggatgtggacgccttcatcagactgaccgtggacgacatgctgaccgacaccgagcggagaatcaagagattcaaggacgaccggaagtccattcggagcgccgacaacaagatgggaaagagaggcttcaagcagatctccacaggcaagctggccgacttcctggccaaggacatcgtgctgtttcagcccagcgtgaacgatggcgagaacaagatcaccggcctgaactaccggatcatgcagagcgccattgccgtgtacgatagcggcgacgattacgaggccaagcagcagttcaagctgatgttcgagaaggcccggctgatcggcaagggcacaacagagcctcatccatttctgtacaaggtgttcgcccgcagcatccccgccaatgccgtcgagttctacgagcgctacctgatcgagcggaagttctacctgaccggcctgtccaacgagatcaagaaaggcaacagagtggatgtgcccttcatccggcgggaccagaacaagtggaaaacacccgccatgaagaccctgggcagaatctacagcgaggatctgcccgtggaactgcccagacagatgttcgacaatgagatcaagtcccacctgaagtccctgccacagatggaaggcatcgacttcaacaatgccaacgtgacctatctgatcgccgagtacatgaagagagtgctggacgacgacttccagaccttctaccagtggaaccgcaactaccggtacatggacatgcttaagggcgagtacgacagaaagggctccctgcagcactgcttcaccagcgtggaagagagagaaggcctctggaaagagcgggcctccagaacagagcggtacagaaagcaggccagcaacaagatccgcagcaaccggcagatgagaaacgccagcagcgaagagatcgagacaatcctggataagcggctgagcaacagccggaacgagtaccagaaaagcgagaaagtgatccggcgctacagagtgcaggatgccctgctgtttctgctggccaaaaagaccctgaccgaactggccgatttcgacggcgagaggttcaaactgaaagaaatcatgcccgacgccgagaagggaatcctgagcgagatcatgcccatgagcttcaccttcgagaaaggcggcaagaagtacaccatcaccagcgagggcatgaagctgaagaactacggcgacttctttgtgctggctagcgacaagaggatcggcaacctgctggaactcgtgggcagcgacatcgtgtccaaagaggatatcatggaagagttcaacaaatacgaccagtgcaggcccgagatcagctccatcgtgttcaacctggaaaagtgggccttcgacacataccccgagctgtctgccagagtggaccgggaagagaaggtggacttcaagagcatcctgaaaatcctgctgaacaacaagaacatcaacaaagagcagagcgacatcctgcggaagatccggaacgccttcgatcacaacaattaccccgacaaaggcgtggtggaaatcaaggccctgcctgagatcgccatgagcatcaagaaggcctttggggagtacgccatcatgaagggatcccttcaatga

SEQ ID NO: 128 - DNA, 20 bp, Homo sapiens
gatgtcgcgtgtcgctgaaa

SEQ ID NO: 129 - DNA, 20 bp, Homo sapiens
caggagccgaccccgttcgt

SEQ ID NO: 130 - DNA, 20 bp, Homo sapiens
gtcggaactattggtcgaaa

SEQ ID NO: 131 - DNA, 20 bp, Homo sapiens
catggcacgtatgaccgctg

SEQ ID NO: 132 - DNA, 20 bp, Homo sapiens
acttataagtatagggaatc

SEQ ID NO: 133 - DNA, 20 bp, Homo sapiens
ggattagttgaaagactgcc

SEQ ID NO: 134 - DNA, 20 bp, Homo sapiens
gtccactaaatgtgacgccc

SEQ ID NO: 135 - DNA, 20 bp, Homo sapiens
gaggacaagccttgacattc

SEQ ID NO: 136 - DNA, 20 bp, Homo sapiens
tgcagtcagatgttatcgtc

SEQ ID NO: 137 - DNA, 20 bp, Homo sapiens
tactggaactcctagatccg

SEQ ID NO: 138 - DNA, 20 bp, Homo sapiens
caatggtcctttgtcaaacg

SEQ ID NO: 139 - DNA, 20 bp, Homo sapiens
tggcaacgtgaacaggtcca

SEQ ID NO: 140 - DNA, 20 bp, Homo sapiens
aacctgaatacatggtaagg

SEQ ID NO: 141 - DNA, 20 bp, Homo sapiens
tactcttgctcatcaagcag

SEQ ID NO: 142 - DNA, 20 bp, Homo sapiens
ctggatgagcaagcgctgta

SEQ ID NO: 143 - DNA, 20 bp, Homo sapiens
tcagagaaggactagaccga

SEQ ID NO: 144 - DNA, 30 bp, Homo sapiens
gctacggggaggaagtgaaagaattcttag

SEQ ID NO: 145 - DNA, 30 bp, Homo sapiens
ggaaaggacctaaagtgtaccgccggaagc

SEQ ID NO: 146 - DNA, 30 bp, Homo sapiens
ggcagtatggacaaaaattcaattcgtcat

SEQ ID NO: 147 - DNA, 30 bp, Homo sapiens
gataaactcaaggagacactaacagcacat

SEQ ID NO: 148 - DNA, 20 bp, Homo sapiens
ctgtcgtagacagcttgtac

SEQ ID NO: 149 - DNA, 20 bp, Homo sapiens
cggtcgcaatctgggtcacg

SEQ ID NO: 150 - DNA, 19 bp, Homo sapiens
gagacacccgcgcagaatc

SEQ ID NO: 151 - DNA, 20 bp, Homo sapiens
cgttctcaagcagcatctcc

SEQ ID NO: 152 - DNA, 20 bp, Homo sapiens
catctccgtattgagtgcga

SEQ ID NO: 153 - DNA, 20 bp, Homo sapiens
cccgttaacattttaattgc

SEQ ID NO: 154 - DNA, 20 bp, Homo sapiens
actgtatagctgtactcggg

SEQ ID NO: 155 - DNA, 20 bp, Homo sapiens
gtggaatgcaggtgtcatac

SEQ ID NO: 156 - DNA, 20 bp, Homo sapiens
catgttggtacatccatccg

SEQ ID NO: 157 - DNA, 20 bp, Homo sapiens
gttgccggtataagagacag

SEQ ID NO: 158 - DNA, 20 bp, Homo sapiens
gacgcaagcattaaaccggg

SEQ ID NO: 159 - DNA, 20 bp, Homo sapiens
ctgggtaaagggtcgcccga

SEQ ID NO: 160 - DNA, 20 bp, Homo sapiens
cggaccgatcacctgagatg

SEQ ID NO: 161 - DNA, 20 bp, Homo sapiens
gccctagcgtcataccacaa

SEQ ID NO: 162 - DNA, 20 bp, Homo sapiens
gcacttactcaacggtctcg

SEQ ID NO: 163 - DNA, 20 bp, Homo sapiens
tactagcctaggaaatactg

SEQ ID NO: 164 - DNA, 20 bp, Homo sapiens
cgtacggagaacttgcagct

SEQ ID NO: 165 - DNA, 20 bp, Homo sapiens
gacgcttatctgactcggcg